(19) United States
(12) Patent Application Publication
(10) Pub. No.: US 2014/0335148 A1
(43) Pub. Date: Nov. 13, 2014

(54) BIOACTIVE SUBSTANCE-CONTAINING NANOFIBERS AND USES THEREOF

(71) Applicants: Ho-Wang Tong, Hong Kong (CN); Lawrence P. Wackett, St. Paul, MN (US); Alptekin Aksan, Minneapolis, MN (US)

(72) Inventors: Ho-Wang Tong, Hong Kong (CN); Lawrence P. Wackett, St. Paul, MN (US); Alptekin Aksan, Minneapolis, MN (US)

(21) Appl. No.: 14/271,958

(22) Filed: May 7, 2014

Related U.S. Application Data
(60) Provisional application No. 61/820,310, filed on May 7, 2013.

(51) Int. Cl. A61K 9/70; D01D 5/00; A61K 47/46; A61K 47/26; A61K 47/42

(52) U.S. Cl. CPC A61K 9/7007; A61K 47/26; A61K 47/42; A61K 47/46; D01D 5/003; D01D 5/0061; D10B 2509/00; B81B 2201/051; D10B 2101/06; D10B 2321/06
USPC 424/443; 514/44 R; 514/1.1; 424/93.6; 424/93.4; 424/93.7; 425/131.5; 264/433

(57) ABSTRACT

An ultrafine fiber comprises a ceramic-based fibrous body and a biologically active substance encapsulated in the body, substantially encapsulated in the body, or surface-attached to the body. In an example, an ultrafine fiber comprises a core comprising a biologically active substance and a ceramic-based shell surrounding or substantially surrounding the core.

### CROSS-REFERENCE TO RELATED APPLICATIONS

This application claims priority under 35 U.S.C. §119(e) to U.S. Provisional Application No. 61/820,310, filed May 7, 2013, which application is incorporated by reference herein in its entirety.

### BACKGROUND

Various biologically active substances (referred to herein as “bioactive substances”), including DNA, RNA, proteins, enzymes, viruses, bacteria and mammalian cells, exhibit a wide spectrum of applications in medicine, biosensing, bioremediation, agricultural delivery, biocatalysis, de novo synthesis of metabolites, and synthesis of recombinant proteins. However, bioactive substances can be difficult to use in their natural state. For example, the uses described above can be difficult to achieve without a means of immobilizing the bioactive substance. For example, without immobilization, enzymes with a useful activity or microorganisms that express that enzyme can be washed away from a desired site of application.

Immobilizing and localizing bioactive substances to a specific location or region of interest can be difficult, particularly in a solution, where the bioactive substance can float freely in solution. Without localizing the bioactive substance, it can be difficult to maintain the activity of the bioactive against various environmental challenges such as changes in temperature, acidity, salinity, or predation by other organisms.

### SUMMARY

The present disclosure describes systems and methods for providing a bioactive substance that has been bioencapsulated in a form that provides for manipulation of the bioencapsulated bioactive substance. The present disclosure also describes ultrafine fibers made by these systems and methods. The systems and methods of the present disclosure can provide for electrospinning the bioactive substance into ultrafine fibers that can be formed into a mat comprising a plurality of the electro spun ultrafine fibers.

The bioactive substance can include DNA, RNA, proteins (such as enzymes), viruses, bacterial cells (which can be either live bacteria or non-living bacteria that can act as an enzyme delivery mechanism), and mammalian cells. The bioactive substance can be combined with an electroprocessed material in the form of fibers or particles or a combination thereof. The electroprocessed material can include polymers, ceramics, metals, metal oxides, or combinations thereof.

In an example, the present disclosure describes ultrafine fibers, and reactive mats made from a plurality of the ultrafine fibers, that each comprises a core and a ceramic-based shell substantially surrounding the core. The shell can comprise a ceramic, such as silica, and a water-soluble material, such as polyvinyl alcohol (PVA). When the ultrafine fibers are used in an aqueous environment, the water-soluble material can dissolve away, leaving behind a porous ceramic shell. A bioactive substance, such as bacterial cells, can be at least partially encapsulated within the core. The pores formed by the dissolution of the water-soluble material can allow molecules from the aqueous environment to diffuse into the core where they can be acted on by the bioactive substance.

The ultrafine fibers described above can be formed using coaxial electrospinning systems and methods that can provide for coaxial electrospinning of a core solution and a shell solution to form the ultrafine fibers. The core solution can form the fiber core and the shell solution can form the fiber shell substantially surrounding the core. The core solution can include a selected bioactive substance and a selected polymer. The shell solution can be a mixture of a ceramic precursor, such as a silica precursor, and a polymer. The ceramic precursor and the polymer can be fed through a microfluidic device. The microfluidic device can be configured to thoroughly mix the two components. The microfluidic device can also be configured to control the reaction time and rate of the two components so that the final mixture can reach a desired viscosity for electro spinning.

In an example, the present disclosure describes an ultrafine fiber comprising a ceramic-based fibrous body and a biologically active substance encapsulated in the body, substantially encapsulated in the body, or surface-attached to the body.

In another example, the present disclosure describes an ultrafine fiber comprising a core comprising a biologically active substance and a ceramic-based shell surrounding or substantially surrounding the core.

In another example, the present disclosure describes a reactive mat or fabric comprising a plurality of ultrafine fibers, each ultrafine fiber comprising a ceramic-based fibrous body and a biologically active substance encapsulated in the body, substantially encapsulated in the body, or surface-attached to the body.

In another example, the present disclosure describes a reactive mat or fabric comprising a plurality of ultrafine fibers, each ultrafine fiber comprising a core comprising a biologically active substance and a ceramic-based shell surrounding or substantially surrounding the core.

In another example, the present disclosure describes a system comprising a ceramic precursor supply system configured to supply a ceramic precursor solution, a polymer supply system configured to supply a polymer solution, a microfluidic device configured to mix and react the ceramic precursor solution and the polymer solution to form an electrospinning solution having a predetermined viscosity, and an electrospinning device configured to electro spin the electrospinning solution to form a fibrous body comprising a ceramic formed from the ceramic precursor solution and a polymer formed from the polymer solution.

In yet another example, the present disclosure describes a method comprising feeding a ceramic precursor solution and a polymer solution to a microfluidic device, mixing the ceramic precursor solution and the polymer solution and reacting the ceramic precursor solution and the polymer solution in the microfluidic device to form an electrospinning solution having a predetermined viscosity, and electrospinning the electrospinning solution to form a fibrous body comprising a ceramic formed from the ceramic precursor solution and a polymer formed from the polymer solution.

These and other examples and features of the present systems and methods will be set forth in part in the following Detailed Description. This Summary is intended to provide an overview of the present subject matter, and is not intended to provide an exclusive or exhaustive explanation. The Detailed Description below is included to provide further information about the present systems and methods.

### BRIEF DESCRIPTION OF THE FIGURES

FIG. 1 is a perspective view of an example system for fabricating ultrafine fibers comprising a bioactive substance-containing core and a ceramic-based shell surrounding the core.

FIG. 2 is a top view of the example system shown in FIG. 1.

FIG. 3 is a conceptual cross-sectional side view of an example ultrafine fiber comprising a bacteria-containing core and a shell surrounding the core.

FIG. 4 is a conceptual cross-sectional view of FIG. 3 taken along line 4-4 in FIG. 3 showing the bacteria encapsulated by the shell.

FIG. 5 is a conceptual cross-sectional view of FIG. 3 taken along line 5-5 in FIG. 3 showing the ultrafine fiber at a point where the bacteria is not present.

FIG. 6 is a conceptual cross-sectional view of an example ultrafine fiber immersed in an aqueous environment, the ultrafine fiber comprising a bacteria-containing core and a shell comprising pores formed due to porogen leaching.

FIG. 7 is a scanning electron microscope (SEM) micrograph of silica and PVA composite fibers fabricated via conventional electrospinning coupled with the use of a microfluidic device.

FIG. 8 is a graph that illustrates the relationship between the viscosity and the concentration of a PVA solution.

FIG. 9 is a transmission electron microscope (TEM) image of an ultrafine fiber comprising a PVA core and a silica and PVA composite shell surrounding the core fabricated via coaxial electrospinning coupled with a microfluidic device.

FIG. 10A and FIG. 10B are fluorescence microscopic images of E. coli bacteria expressing green fluorescent protein (GFP) encapsulated in silica and PVA composite ultrafine fibers fabricated via conventional electrospinning coupled with a microfluidic device, where the electrospinning duration was 3 min in FIG. 10A and 30 min in FIG. 10B.

FIG. 11 is an SEM micrograph of an E. coli bacterium encapsulated in a silica and PVA composite fiber fabricated via conventional electrospinning coupled with a microfluidic device.

FIG. 12 is a TEM image of an E. coli bacterium encapsulated in a silica and PVA composite fiber fabricated via conventional electrospinning coupled with a microfluidic device.

FIG. 13 is a confocal microscopic image of E. coli bacteria expressing GFP encapsulated in a core-shell fiber with a silica and PVA composite shell fabricated via coaxial electrospinning coupled with a microfluidic device.

FIG. 14 is an SEM image of core-shell fibers having an E. coli-containing core and a silica and PVA composite shell surrounding the core fabricated via coaxial electrospinning coupled with a microfluidic device

FIG. 15 shows a Fourier transform infrared spectroscopy (FTIR) spectra of silica and PVA composite fibers and E. coli-containing silica and PVA composite fibers fabricated via conventional electro spinning.

FIG. 16 is a calibration curve for estimating a mass percentage of E. coli in electrospun silica and PVA composite fibers.

### DETAILED DESCRIPTION

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a concentration range of “about 0.1% to about 5%” should be interpreted to include not only the explicitly recited concentration of about 0.1 wt. % to about 5 wt. %, but also the individual concentrations (e.g., 1%, 2%, 3%, and 4%) and the sub-ranges (e.g., 0.1% to 0.5%, 1.1% to 2.2%, and 3.3% to 4.4%) within the indicated range.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods of manufacturing described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

### DEFINITIONS

The singular forms “a,” “an” and “the” can include plural referents unless the context clearly dictates otherwise.

The term “about” can allow for a degree of variability in a value or range, for example, within 10%, or within 5% of a stated value or of a stated limit of a range.

The term “independently selected from” refers to referenced groups being the same, different, or a mixture thereof, unless the context clearly indicates otherwise. Thus, under this definition, the phrase “X1, X2, and X3 are independently selected from noble gases” would include the scenario where, for example, X1, X2, and X3 are all the same, where X1, X2, and X3 are all different, where X1 and X2 are the same but X3 is different, and other analogous permutations.

The term “cell” as used herein refers to bacteria, mammalian cells, archaea, protist, or fungi.

The term “microorganism” as used herein refers to bacteria, archaea, protist, or fungi.

The term “bioactive substance” refers to one or more microorganisms, cells, or enzymes such as enzymes within a cell or microorganism or enzymes not within a cell or microorganism (free enzymes). Examples of bioactive substances include enzymes, macromolecules, and non-mammalian cells, such as for example bacteria, archaea, protists, or fungi.

The term “pore” as used herein refers to a depression, slit, or hole of any size or shape in a solid object. A pore can run all the way through an object or partially through the object. A pore can intersect other pores.

The term “silica” as used herein can refer to silicon dioxide (SiO₂) of any particle size, shape, particle size distribution, shape distribution and surface functionality, including chemically treated silicas. It can also refer to a polysiloxane.

### DESCRIPTION

The present disclosure describes ultrafine fibers, reactive mats and fabric made from a plurality of the ultrafine fibers, that each comprises an electroprocessed material and a bioactive substance. The electroprocessed material can include any polymer, ceramic, metal, metal oxide, or combination thereof, wherein the electroprocessed material can be electro spun to form an ultrafine fiber comprising the bioactive substance being at least partially encapsulated in the electroprocessed material. In an example, the fibers and reactive mats formed therefrom can include a core-shell type fiber with a core comprising the bioactive substance and a ceramic-based shell substantially surrounding the core. The shell can comprise a ceramic, such as silica, and a water-soluble material, such as polyvinyl alcohol (PVA). When the ultrafine fibers are used in an aqueous environment, the water-soluble material can dissolve away, leaving behind a porous ceramic shell. A bioactive substance, such as bacterial cells, can be at least partially encapsulated within the core. The pores formed by the dissolution of the water-soluble material can allow molecules from the aqueous environment to diffuse into the core where they can be acted on by the bioactive substance.

The present disclosure also describes systems and methods to form the ultrafine fibers described above. The systems and methods can provide for coaxial electro spinning of a core solution and a shell solution to form the ultrafine fibers. The core solution can form the fiber core and the shell solution can form the fiber shell substantially surrounding the core. The core solution can include a selected bioactive substance and a selected polymer. The selected polymer in the core can serve as a thickener to adjust the viscosity of the core solution that facilitates the drawing of the core solution into a fiber core by a shear force at the interface of the core solution and the shell solution during electrospinning. The shell solution can be a mixture of a ceramic precursor, such as a silica precursor, and a polymer that is mixed within a microfluidic device. The microfluidic device can mix the two components thoroughly and can also precisely control the reaction time and rate of the two components so that the final mixture can reach and sustain a desired viscosity that can provide for continuous or substantially continuous electro spinning. The selected polymer in the shell can serve as a thickener and can also serve as a porogen material to leach from the shell when the reactive mat is exposed to aqueous environments. The polymer can further be water soluble so that an organic solvent is not necessary, e.g., so that the polymer can also participate in the reaction that forms the fibers in such a way that an organic solvent, such as ethanol, is not necessary for continuous or substantially continuous electrospinning. In an example, the polymer can be at least partially cross-linked with the ceramic precursor, e.g., the silica precursor, in the microfluidic device in a controlled manner so that the desired viscosity of the solution can be maintained.

Ultrafine fibers formed using the systems and methods described herein, and reactive mats formed using those ultrafine fibers, can be used, for example, to provide prolonged release of desired enzymes from encapsulated bacteria. The ultrafine fibers and reactive mats can also be used for diffusion of contaminants from a medium outside the fibers to encapsulated bioactive substance through pores or the thin wall of the ultrafine fibers, resulting in biotransformation of the contaminants into non-toxic products.

Ultrafine fibers formed using the systems and methods described herein, and reactive mats formed using those ultrafine fibers, can be used, for example, in medicine including but not limited to tissue engineering and drug delivery, bio sensing, bioremediation, agricultural delivery, biocatalysis, de novo synthesis of metabolites, and even synthesis of recombinant proteins.

For example, ultrafine fibers and reactive mats or fabrics formed from the fibers can be used for bioremediation of undesirable contaminants, for example herbicides and pesticides such as atrazine (described in more detail below). Ultrafine fibers and reactive mats or fabrics formed from the fibers can also be used as bioreactors within aqueous or other media. The use of the fibers or reactive mats or fabrics for bioremediation, or for other reactive applications, can be beneficial because the fibers can have a very large surface area relative to the mass of the bioactive substance and can also have a very high ratio of the mass of the bioactive substance to the ceramic or other support material (e.g., silica or silica gel network) of the fiber. In an example, a reactive mat or fabric of a plurality of the ultrafine fibers can be used to perform clean-up of a spill of an undesirable compound, e.g., by placing the reactive mat or fabric onto a spill, such as a spill on the soil, so that the reactive mat or fabric can absorb and degrade the undesirable compound. Alternatively, a reactive mat or fabric can be placed on or in an aqueous medium (e.g., water) with a spilled undesirable compound thereon, e.g., in the form of a chemical slick on the top of the aqueous medium.

Fibers or reactive mats or fabrics according to the present disclosure can also be used for treating of gases, e.g., to remove toxic or otherwise undesirable gases from a volume or gas stream (such as hydrogen sulfide, hydrogen cyanide, etc.), or to remove noxious odors from the gas volume or gas stream. In another example, the fibers or reactive mats or fabrics can be used to form a gaseous bioreactor to transform natural gas (methane) into valuable chemicals using bacteria or other bioactive substances as the catalysts. Methane is now cheaper and more readily available than glucose and sucrose, which have traditionally been the major carbon sources in biotechnology applications. Methane is poorly soluble in water such that it has been challenging to design fermentor biotechnology because process throughput is limited by the mass transfer of methane from gas to liquid phase in feeding the bacteria or other bioactive substances. This can greatly hold back yield per unit time. Reactive mats, fabrics, or other structures formed at least in part from the ultrafine fibers described herein can provide for highly concentrated bacteria or other bioactive substances to metabolize methane, and potentially make a product.

Fibers or reactive mats or fabrics according to the present disclosure can also be used as an indicator structure to indicate the presence of a particular compound, such as a contaminant. In an example, the ultrafine fibers can include an encapsulated bioactive substance that can fluoresce in the presence of a contaminant. For example, the ultrafine fibers that form the reactive mat or fabric can include a bacteria that expresses an enzyme or other compound that fluoresces in the presence of carbon monoxide. The reactive mat or fabric can be used to fluoresce when exposed to carbon monoxide leaking from a pipe, a valve, or the like. An indicator reactive mat or fabric can also be configured to degrade or convert the compound in addition to indicating its presence.

Ultrafine fibers formed using the systems and methods described herein, and reactive mats or fabrics formed using those ultrafine fibers, can be used, for example, for encapsulation of different types of bioactive substances (e.g., DNA, RNA, proteins, viruses, bacteria and mammalian cells) into ultrafine fibers having a ceramic-based body or a ceramic-based shell.

Bioactive substances can often exhibit properties that can be useful in a wide variety of applications. The metabolic functionality of bioactive substances can have extensive applications in biotechnology (e.g. biosensing, biocatalysis, bioremediation, and bioreactors), medicine (e.g. regenerative medicine, tissue engineering, and recombinant protein production), and in new hybrid materials with improved functional and structural properties. One example of a useful property exhibited by bioactive substances is the catalytic activity exhibited by enzymes. For example, the catalytic activity of enzymes can be useful for chemical transformations on a small-scale in a chemistry lab, in large-scale industrial chemical manufacturing or purification operations, in agricultural settings, in food products, and in water treatment.

Bioactive substances can be difficult to utilize in their natural state. The useful applications described above are difficult to achieve without some way to immobilize the bioactive substance. Without immobilization, enzymes with a useful activity or microorganisms that express an enzyme with useful activity can be easily washed away from a desired site of application. Immobilization can allow repeated use without requiring separation and purification of the enzyme or addition of new catalysts. However, to enable practical application of their useful activities, successful immobilization of microorganisms depends on a highly biocompatible encapsulation material of sufficient mechanical robustness that permits the entry of small molecules such as O₂, nutrients, electrolytes, and exit of toxic metabolites, hormones, and other bioactive compounds.

Encapsulation of the bioactive substance, also referred to as bioencapsulation, is one method for immobilizing the bioactive substance. Immobilization via bioencapsulation can prevent the bioactive substance from being washed away in an aqueous environment or can greatly reduce the rate at which the bioactive substance is washed away or leached into the aqueous environment. Encapsulation can also separate the bioactive substance from the environment outside the encapsulating structure and can protect the bioactive substance from the environment or can protect the environment from the bioactive substance, or both. Encapsulation can also provide for a greater density of the bioactive substance beyond what can typically be achieved in nature, therefore providing for an increased chemical reaction rate using the encapsulated bioactive substance. In some examples, encapsulation of the bioactive substance can also allow for control of the location and concentration of the bioactive substance by controlling the location of the structures that encapsulate the bioactive substance, e.g., the location of fibers such as the core-shell fibers described below, or by controlling the concentration of the bioactive substance in the encapsulating structures, or both.

While the immobilization of microorganisms, such as via encapsulation of the bioactive substance, has been attempted using various substrates and techniques, traditional materials used for cell encapsulation have limited the development of biotechnology and medical applications due to the instability of the bioactive substance over long periods of time. Problems with stability can include limited or no catalytic activity expressed by an immobilized enzyme or by an immobilized microorganism that expresses the particular enzyme. Problems with stability can also sometimes include death of the microorganism, although short- or long-term survival or viability of the microorganism is not always required in all applications. Additionally, the mechanical properties of polymeric synthetic and natural materials used for cell encapsulation can be drastically altered by the metabolically active encapsulated cells.

In the past, many problems have been experienced in immobilization of macromolecules, specifically, microorganisms. For example, when immobilizing microorganisms using silica nanoparticles, proteins expressed and released by the microorganisms can be adsorbed into the silica nanoparticles or silica gel networks, which can cause denaturation and aggregation of the adsorbed proteins and therefore loss of structure and catalytic activity. Traditional microorganism immobilization protocols can also lead to adsorption and denaturation when temperatures are increased. Traditional immobilization protocols can also result in the product or use of alcohols or other chemicals during a sol-gel reaction, which can create an environment that is toxic to the encapsulated cell or other bioactive substance. When encapsulation procedures include hydrolysis or condensation steps, the procedure can require additional steps to remove the byproducts of the hydrolysis or condensation reactions. When encapsulation procedures include the use of colloidal precursors such as sodium or potassium silicate, the removal of the sodium or potassium ions can be required.

Bioencapsulation can involve encapsulation, and hence immobilization, of bioactive substance in a permeable medium. Bioencapsulation can also provide for growth inhibition and controlled diffusion limitations for the nutrients, which result in low reaction rates. For example, when growth is inhibited, an increase in the metabolic activities of the microbes and enhanced biodegradation can occur because the energy that would have been otherwise spent for proliferation is now made available for other functions, such as degradation. Furthermore, the inhibition of diffusion of certain chemicals can be beneficial, for example to protect the bioactive substance against toxins and osmotic stress.

Electrospinning is a technology capable of forming ultrafine fibers. Electrospinning is viewed as a promising approach to minimize diffusion limitations because bioactive substances can be encapsulated into thin-walled electrospun ultrafine fibers. When using electrospun ultrafine fibers as an encapsulating material for bioencapsulation, the fibers can be biocompatible so that the activity of the encapsulated bioactive substances can be maintained. The fibers can also be made insoluble so that the fibers can remain intact in aqueous environments. Moreover, a majority of bioactive substances can be encapsulated into the fibers rather than attached onto the surface of the fibers. These capabilities can depend on the selected bioactive substance for the ultrafine fiber and on the ultrafine fiber fabrication technique.

Conventional electrospinning technique can be employed to encapsulate bioactive substances into fibers made of water-insoluble materials such as polycaprolactone and Pluronic F127 dimethacrylate. The fabrication process of conventional electrospinning, however, can involve the use of organic solvents such as chloroform, dimethylformamide, and dichloromethane that are usually toxic to bioactive substances. Some studies have been reported that made electrospun fibers with encapsulated bioactive substances without using organic solvents, the fibers were made of water-soluble materials. Several studies have investigated the use of organic solvents with low toxicity or coaxial electrospinning to minimize the contact of organic solvent molecules with bioactive substances. Despite these efforts, electrospinning of bioactive substances into water-insoluble fibers that maintain the bioactivity of the bioactive substances without using organic solvents is still a challenge.

Coaxial electrospinning is similar to conventional electrospinning except the configuration of the spinneret. For conventional electrospinning, the spinneret is usually includes a single conduit with a single orifice. For coaxial electrospinning, the spinneret can comprise an inner conduit positioned radially inside of an outer conduit, such as an inner needle or capillary, e.g., a core conduit, positioned concentrically inside a larger outer need needle or capillary, e.g., a shell conduit. A first solution can be supplied to the inner or core conduit and is referred to herein as a “core solution.” A second solution can be supplied to the outer or shell conduit and is referred to herein as a “shell solution.” The shell solution can be drawn by an electric force applied to the outer conduit to form a fiber shell. The core solution can be drawn by a shear force at an interface between the core solution and the shell solution, forming a fiber core that is substantially surrounded by the fiber shell. An alternative setup for coaxial electrospinning can be composed of a conduit, such as a needle or capillary, carrying core solution being inserted directly into a cone formed from the shell solution being electrically drawn from the shell conduit.

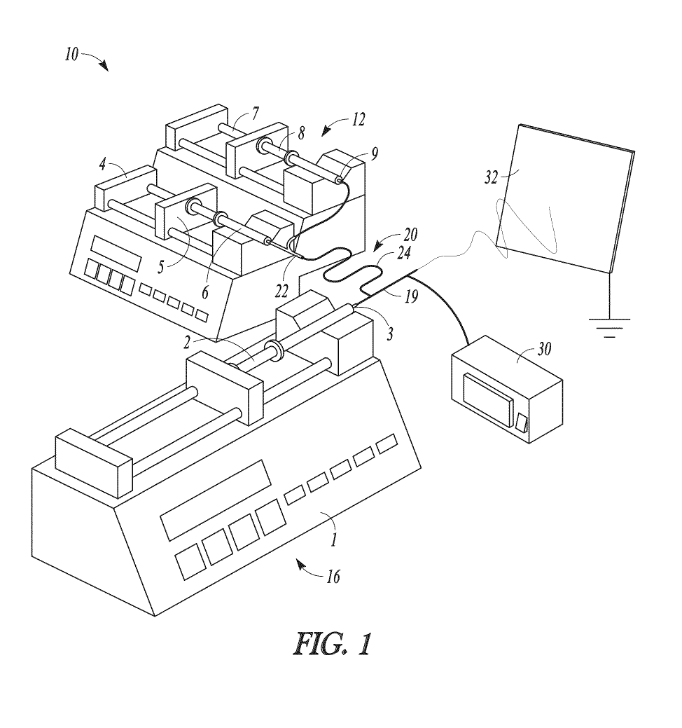

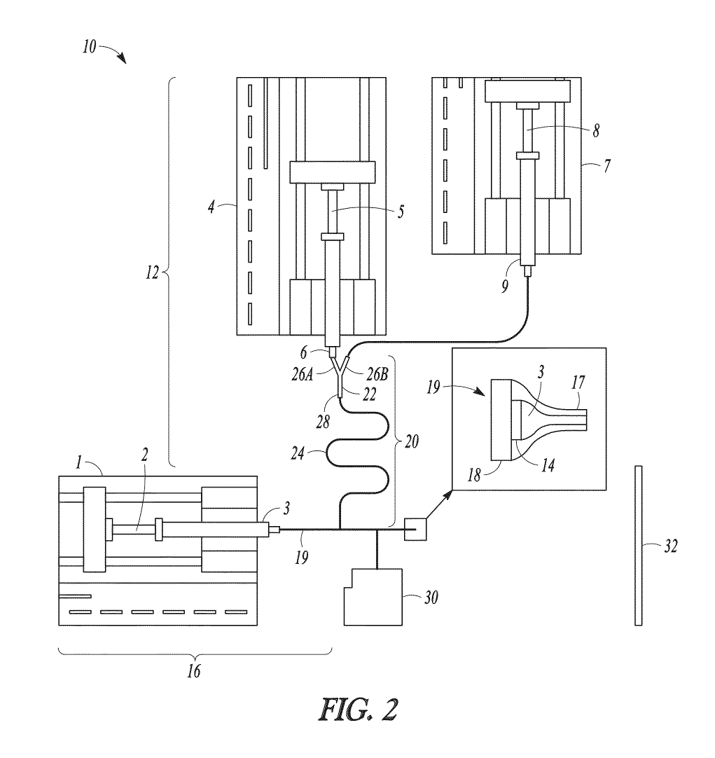

FIGS. 1 and 2 show schematic diagrams of an example electrospinning system 10 configured to encapsulate bioactive substances into ceramic-based ultrafine fibers. FIG. 1 shows a perspective view of the example electrospinning system 10, while FIG. 2 shows a top view of the example electrospinning system 10. The system 10 can include a core solution delivery system 12 for delivering a core solution 3 to an inner conduit 14, also referred to herein as a core conduit 14, and a shell solution delivery system 16 for delivering a shell solution 17 to an outer conduit 18, also referred to herein as a shell conduit 18.

The core solution 3 can include a bioactive substance, such as DNA, RNA, proteins, viruses, live bacteria or mammalian cells, mixed with a polymer solution, such as a thickener solution to form a mixture of the core solution 3. In an example, the thickener can include a solution of PVA, PEO, PEG, gelatin, dextran, or carbohydrate.

In an example, the bioactive substance of the core solution 3 can include a bacterium, such as Escherichia coli (E. coli) that expresses a particular enzyme of interest. In an example, the bioactive substance can include E. coli expressing green fluorescent protein (GFP) or E. coli expressing atrazine chlorohydrolase (AtzA). For example, reactive mats comprising fibers encapsulating E. coli expressing AtzA can be used for bioremediation or, more specifically, can achieve prolonged biotransformation of atrazine into non-toxic products. In an example, the bacteria (e.g. E. coli) can be loaded into the core solution 3 at less than or equal to about 0.5 g E. coli per mL of core solution 3.

The core solution delivery system 12 can include a syringe 2 into which the core solution 3 can be loaded. The syringe 2 can be connected to the core conduit 14. The core conduit 14 can be placed radially within the shell conduit 18, for example concentrically inside the shell conduit 18, to form a nested conduit assembly 19. The core solution delivery system 12 can also include a flow control device, such as a syringe pump 1, to control a flow rate of the core solution 3 being fed to the core conduit 14.

The shell solution 17 can comprise a mixture of a polymer solution 6, such as a thickener or porogen solution 6, and a ceramic precursor solution 9. For example, the ceramic precursor solution 9 can include a silica precursor solution 9. Examples of silica precursor solutions include, but are not limited to tetramethylortho silicate (TMOS), tetramethylortho silicate (TEOS), tetrakis(2-hydroxyethyl)ortho silicate (THEOS), methyldiethoxysilane (MDES), 3-(glycidoxypropyl)triethoxysilane (GPMS), 3-(trimethoxysilyl)propylacrylate (TMSPA), n-(3-triethoxysilylpropyl)pyrrole (TESPP), vinyltriethoxysilane (VTES), methacryloxypropyltriethoxysilane (TESPM), silica nanoparticles (e.g. Ludox or Nyacol), sodium silicate (e.g., 27% silicic acid and 10% NaOH, and diglycerylsilane (DGS). In some examples, structure modifiers can be used, such as, but not limited to, methyltriethoxysilane (MTMOS), trimethylmethoxysilane (TMMS), ethyltriethoxysilane (TEES), n-propyltriethoxysilane (TEPS), n-butyltriethoxysilane (TEBS), 3-aminopropyltriethoxysilane (APTS), 3-(2,4-dinitrophenylamino)propyltriethoxysilane, mercaptopropyltriethoxysilane, 3-(2-aminoethylamino)propyltriethoxysilane, isocyanatopropyltriethoxysilane, hydroxyl-terminated polydimethylsiloxane (PDMS), triethoxysilyl-termonated polydimethylsiloxane (PDMS), methyltriethoxysilane (MTES), triethoxysilyl-terminated poly(oxypropylene). The polymer solution 6 can comprise a solution of PVA, PEO, PEG, gelatin, dextran, or carbohydrate.

In an example, the inner conduit 14 can have an inner diameter of from about 0.2 mm (e.g., a 27-gauge needle) and about 0.5 mm (e.g., a 21-gauge needle), such as about 0.4 mm (e.g., a 22-gauge needle). In an example, the outer conduit 18 can have an inner diameter of from about 0.84 mm (e.g., an 18-gauge needle) to about 1.6 mm (e.g., a 14-gauge needle).

As noted above, the shell of the ultrafine particles can include a ceramic-based material. Examples of ceramic-based materials that the final shell can comprise include, but are not limited to, one or more of a silica-based material and a bioactive glass-based material. The ceramic-based material can comprise a ceramic gel network, such as a silica gel network or a bioactive glass gel network. The ceramic gel network can be at least partially cross-linked with the polymer of the shell solution 17. Silica-based materials can be a desirable candidate material for bioencapsulation because of its biocompatibility, mechanical robustness, thermal stability and relative inertness.

To form the ceramic-based material of the shell, the shell solution 17 can include a ceramic precursor. For example, if a silica-based shell is desired, TMOS can be used as a silica precursor. Other silica precursor materials can be used, as described above. For the sake of brevity, the remainder of the present disclosure will describe the formation of a silica-based shell using TMOS as the ceramic precursor. A person of ordinary skill in the art will understand that other biocompatible ceramic-based materials can be used.

Silica ultrafine fibers have been made by electrospinning blend solutions of polymer and colloidal silica nanoparticles, followed by selective removal of the polymer component by calcination to form the fiber shape of silica nanoparticle assembly. But the high temperature (e.g., around 450° C.) involved in the process limits its application for bioencapsulation because the high temperature can tend to deactivate the bioactive substance, e.g., by killing bacterial or mammalian cells or denaturing or destroying the bioactive substance. Sol-gel electrospinning has also been used to form silica ultrafine fibers without application of high temperatures. However, sol-gel electrospinning generally requires the use of ethanol or other organic solvents, which can be incompatible with bioactive substances. Sol-gel electrospinning can also result in substantially continuously changing viscosity of the solution, which is not favorable for large-scale, continuous formation of fibers. Therefore, neither electrospinning using solutions of polymer and colloidal silica nanoparticles nor sol-gel electrospinning is conducive to the formation of fibers comprising encapsulated bioactive substances that retain their bioactivity during formation of the fibers. Successful encapsulation of bioactive substances into ultrafine silica fibers while maintaining the activity of the substances, therefore, can be very difficult to achieve.

The shell solution 17 can also include a polymer provided in the polymer solution 6 that can be mixed with the ceramic-based material. The polymer can include a natural polymer or a synthetic polymer. The polymer can act as thickener material or a porogen, or both. In an example, the polymer can comprise a water-soluble polymer so that when the ultrafine fiber formed from the ceramic-based material and the polymer is exposed to an aqueous environment, the water-soluble polymer can dissolve into the aqueous environment to leave behind a plurality of pores in the ceramic-based shell. Examples of natural polymers that can be used in the shell solution 17 include, but are not limited to, one or more of collagen, elastin, gelatin, fibrinogen, fibrin, alginate, cellulose, silk fibroin, chitosan and chitin, hyaluronic acid, dextran, wheat gluten, polyhydroxyalkanoates, and laminin. Examples of synthetic polymers that can be used in the shell solution 17 include, but are not limited to, one or more of polyvinyl acid (PVA), polyethylene oxide (PEO), polyethylene glycol (PEG), nylon, polycarbonate (PC), polybutylene terephthalate (PBT), polyurethane (PU), poly(ethylene vinyl acetate) (PEVA), polycaprolactone (PCL), polyglycolic acid (PGA), polylactic acid (PLA), and poly(lactic-co-glycolic acid) (PLGA). In an example, the thickener material and porogen comprises polyvinyl alcohol (PVA), wherein the PVA can also act as a porogen.

Further description of materials that can be used to form the core solution 3 or the shell solution 17 are described in published PCT application WO2012/116013, filed under the PCT on Feb. 22, 2012, published on Aug. 30, 2012, which claims priority to U.S. Provisional Patent Application No. 61/445,204, filed on Feb. 22, 2011, the disclosures of which are incorporated by reference herein as if reproduced in their entirety.

Other materials can also be included in the shell solution 17, such as metals or metal oxides. Examples of metals or metal oxides that can be included in the shell solution 17 include, but are not limited to, one or more of cobalt, iron, copper, copper oxide, and titanium dioxide.

A polymer solution 6 including a thickener, such as PVA, introduced into the shell solution 17 can be configured so that the shell solution 17 achieves a desired viscosity that is optimized for electro spinning. The viscosity of the final shell solution 17 can depend on one or more of the reaction time of the ceramic precursor solution 9 and the polymer solution 6, the reaction rate of the ceramic precursor solution 9 and the polymer solution 6, and the concentration polymer in the polymer solution 6. The polymer can also serve as a porogen, as PVA can do, so that the polymer component of the reactive mats can dissolve in aqueous environments, thus increasing the porosity and the permeability of the fibers and hence decreasing the resistance to diffusion.

The shell solution delivery system 16 can include a syringe 5 into which the polymer solution 6 can be loaded, a syringe 8 into which the ceramic precursor solution 9 can be loaded, and a microfluidic device 20 that can provide for mixing and reaction of the ceramic precursor solution 9 and the polymer solution 6. The shell solution delivery system 16 can also include a flow control device to control a flow rate of the polymer solution 6, such as a syringe pump 4, and a flow control device to control a flow rate of the ceramic precursor solution 9.

The microfluidic device 20 can combine the polymer solution 6 and the ceramic precursor solution 9 to form the shell solution 17 that can be fed to the shell conduit 18. In an example, the microfluidic device 20 can include a generally Y-shaped three-way adapter 22 and tubing 24 extending downstream from the generally Y-shaped three-way adapter 22. The syringe 5 containing the polymer solution 6 can be connected to a first inlet 26A of a Y-shaped three-way adapter 22, and the syringe 8 containing the ceramic precursor solution 9 can be connected to a second inlet 26B of the Y-shaped three-way adapter 22. The outlet 28 of the Y-shaped three-way adapter 22 can be connected to an inlet of the tubing 24. An outlet of the tubing 24 can be connected to the shell conduit 18.

The microfluidic device 20 can be configured to modulate the chemistry or one or more physical properties, or both, of the electro spinning liquid. For example, the microfluidic device 20 with inlets of the ceramic precursor solution 9 and the solution 6 can be connected to the shell conduit 18 of the coaxial electro spinning system 10. The microfluidic device 20 can thoroughly mix the ceramic precursor solution 9 with the polymer solution 6 before the mixture solution is fed to the shell conduit 18, and thus before the mixture solution is drawn by electric force at the orifice of the shell conduit 18. The microfluidic device 20 can be configured to precisely control a reaction, a reaction rate, or both, of the ceramic precursor solution 9 and the polymer solution 6. The microfluidic device 20 can initiate the participation of the ceramic precursor of the ceramic precursor material 9 in cross-linking of the polymer material of the polymer solution 6 to form a fluid with a predetermined viscosity that can be ideal for electrospinning, as described in more detail below.

The tubing 24 of the microfluidic device 20 can be configured with length that is selected to provide for a desired viscosity of the resulting shell solution 17 that is optimized or substantially optimized for electro spinning of continuous fibers.

The materials of the core solution 3 and the shell solution 17 described herein can be difficult to electro spin because they typically form fluids with continuously changing viscosity. A fluid with a continuously changing viscosity, such as the shell solution 17 and the core solution 30, can typically only be drawn into a fiber via electro spinning if the solution is within a narrow range of viscosities, sometimes referred to herein as an “optimal viscosity,” an “optimum viscosity,” or a “predetermined viscosity.” Within the optimal viscosity range, the electric force of the electro spinning device (described in more detail below) can be high enough to initiate a jet from the solution without breaking the jet, which then becomes a continuously spun ultrafine fiber.

If the viscosity of the solution is too high, the electric force will not be strong enough to initiate a jet, resulting in no fiber formation. On the contrary, when the viscosity of the solution is too low, the jet can break into small droplets while the electric force is stretching the jet, resulting in formation of droplets instead of fibers. Therefore, for a solution having continuously changing viscosity (as in the case of a gelling sol or a polymerizing solution) is subjected to conventional electro spinning, a mixture of droplets (when the viscosity is below the optimum range) and fibers (when the viscosity reaches the optimum range) is obtained. Further, the electro spinning process cannot continue when the viscosity is above the optimum range (and the electro spinning needle is clogged). Solutions that are a mixture of ceramic precursor, such as a silica precursor solution, and a thickener polymer solution, such as PVA polymer or other polymers described herein, can result in a solution of constantly changing viscosity.

The viscosity can be controlled by selecting the length because the length of the tubing 24 can provide for control the reaction of the ceramic precursor solution 9 and the polymer solution 6 so that the reaction time or the reaction rate, or both, can be optimized, e.g., to provide for the desired viscosity of the shell solution 17. The length of the tubing 24 can also be selected so that the ceramic precursor solution 9 and the polymer solution 6 can be thoroughly mixed, which can also provide for the desired viscosity of the shell solution 17, which can provide for uniformity of the resulting gel.

In an example, the tubing 24 and other components of the microfluidic device 20 can be configured so that the polymer precursor solution 6 (e.g., a PVA solution) and the ceramic precursor solution 9 (e.g., a silica precursor solution) have a predetermined residence time within the microfluidic device 20 (e.g., within the tubing 24) of from about 5 minutes to about 15 minutes, such as from about 8 minutes to about 10 minutes, for example about 9 minutes. In general, if the residence time of the polymer solution 6 and the ceramic precursor solution 9 in the microfluidic device 20 is reduced, the ratio of the ceramic precursor solution to the polymer solution, by volume, can be reduced, or the concentration of the polymer in the polymer solution (e.g., the mass of PVA per volume of the PVA solution) can be increased, or both. Conversely, if the residence time in the microfluidic device is increased, generally, the ratio of the ceramic precursor solution to the polymer solution, by volume, can be increased, or the polymer concentration in the polymer solution can be decreased, or both.

Other factors that can affect the viscosity of the shell solution 17 include, but are not limited to, the volume ratio of the ceramic precursor solution 9 to the polymer solution 6 in the shell solution 17 and the concentration of the polymer (e.g., the thickener/porogen) in the polymer solution 6. These parameters can also be controlled and optimized to provide for a desired viscosity to provide for electro spinning of continuous fibers. The volume ration of the ceramic precursor solution 9 to the polymer solution 6 can be controlled by controlling the flow rates of the solutions 6, 9 using the syringe pumps 4 and 7, respectively.

In an example, the volume ratio of the ceramic precursor solution, e.g., a TMOS solution, to the polymer solution, e.g., a PVA solution, can be from about 1 (e.g., a 1:1 ratio, by volume, of TMOS to PVA) to about 2 (e.g., a 2:1 ratio, by volume of TMOS to PVA), for example about 4:3 (e.g., a 4:3 ratio, by volume, of TMOS to PVA). In general, if the ratio of the ceramic precursor solution to the polymer solution is reduced, the reaction or mixing time (or both) within the microfluidic device 20 will be decreased, or the concentration of the polymer in the polymer solution (e.g., the mass of PVA per volume of the PVA solution) will be increased, or both. Conversely, if the ratio of the ceramic precursor solution to the polymer solution is increased, then the reaction or mixing time (or both) can be increased, or the polymer concentration in the polymer solution can be decreased, or both.

In an example, the flow rate of the shell solution 17 (e.g., the mixture of the polymer solution 6 and the ceramic precursor solution 9) fed to the outer conduit 18 can be from about 10 μL/min to about 30 μL/min, such as about 20 μL/min. The corresponding flow rates of the polymer solution 6 and the ceramic precursor solution 9 can be adjusted based on the desired flow rate of the shell solution 17 and the desired ratio, by volume, of the ceramic precursor solution 9 to the polymer solution 6.

In an example, a concentration of the polymer (e.g., PVA) in the polymer solution 6 can be from about 8% (g/mL) to about 28%, such as about 18%. In general, if the concentration of the polymer in the polymer solution 6 is reduced, the reaction or mixing time (or both) within the microfluidic device 20 can be increased, or the ratio of the ceramic precursor solution 9 to the polymer solution 6, by volume, can be decreased, or both. Conversely, if the concentration of the polymer in the polymer solution 6 is increased, then the reaction or mixing time (or both) can be decreased, or the ratio of the ceramic precursor solution 9 to the polymer solution 6, by volume, can be increased, or both.

The optimal viscosity can depend on the materials being electro spun. For example, for a shell solution 17 of a silica precursor (TMOS) and PVA, the optimal viscosity range of the solution 17 can be from about 2600 cP (±about 118 cP) to about 5413 cP (±about 255 cP), such as about 4173 cP (±about 686 cP).

A voltage is applied to the shell conduit 18 by a power supply 30, such as a high-voltage power supply 30, so that the shell solution 17 can be drawn by electric force. The core solution 3 can be drawn by a shear force at the interface of the core solution 3 and the shell solution 17 caused by the advancing shell solution 17. As the process continues, reactive mats comprising the resulting core-shell type fibers with a bioactive substance-containing core and a ceramic-based shell surrounding the core can be collected by a conductive plate 32, which is placed at a distance from a tip of the nested conduits 14, 18.

Coaxial electrospinning, when coupled with the microfluidic device 20, can be an efficient technique of encapsulating a bioactive substance, such as enzymes or bacteria expressing an enzyme, into ceramic-based ultrafine fibers without compromising the enzymatic activity of the encapsulated enzyme or bacteria. For example, coaxial electrospinning, when coupled with the microfluidic device 20, can allow for encapsulation of bioactive substance into ceramic-based ultrafine fibers at temperatures that do not interfere with the bio activity of the bioactive substances. Coaxial electrospinning in conjunction with the microfluidic device 20 can also allow for encapsulation of bioactive substance into ceramic-based ultrafine fibers without the use of ethanol or other organic solvents while maintaining a viscosity of the solution that is optimized for continuous formation of the ultrafine fibers via electro spinning. This is a feature that is not attainable with previous electrospinning systems. As noted above, other methods of electrospinning ceramics like silica either require high temperatures or the use of ethanol or other organic solvents that would deactivate bioactive substance. In contrast, coaxial electrospinning with the system 10, including the use of the microfluidic device 20, can provide for encapsulation of the bioactive substance in ceramic-based ultrafine fibers while also preserving their bioactivity.

Although FIGS. 1 and 2 show the microfluidic device 20 being used in a coaxial electrospinning system 10, a microfluidic device according to the present description can also be used with a conventional electrospinning system, e.g., where a single-layer of material forms the fiber rather than the core-shell type of fiber described above. The use of a microfluidic device with a conventional electrospinning system can still provide for precise control of one or more of reaction time or reaction rate of the components of the electrospinning mixture in order to control the viscosity of the electrospinning mixture to be within an optimal viscosity range. The conventional electrospinning system with a microfluidic device can also produce ceramic-based ultrafine fibers comprising bioactive substance having both surface-attached bioactive substance and encapsulated bioactive substance.

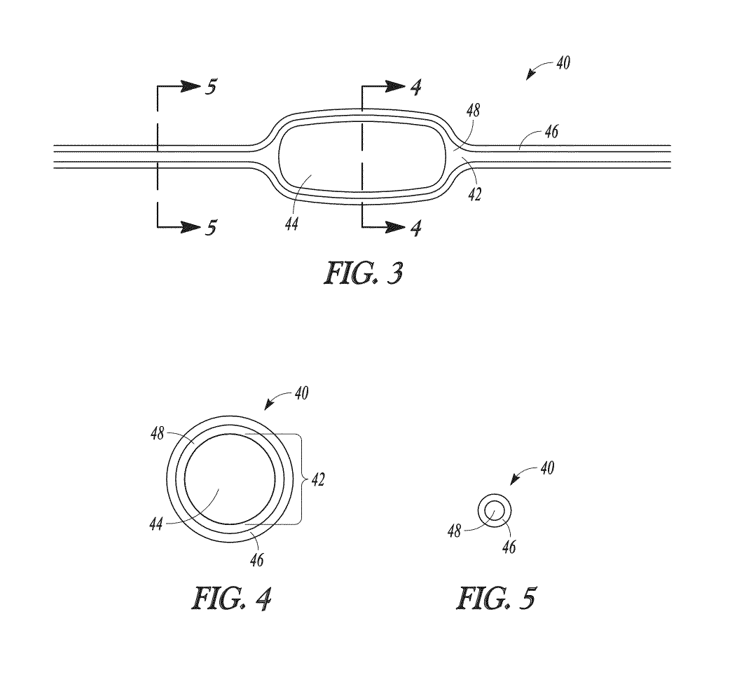

FIGS. 3-5 illustrates an example of a core-shell type ultrafine fiber 40 with a core 42 comprising encapsulated bioactive substance 44, such as bacteria 44, and a shell 46 surrounding the core 42. The core 42 can serve as a reservoir for the bioactive substance 44 within the fiber 40. In an example, the core 42 can comprise a polymer 48 that can at least partially encapsulate the bioactive substance 44 within the core 42. In an example, the polymer 48 can include at least one of PVA, PEO, PEG, gelatin, dextran, and carbohydrate.

The shell 46 can comprise a ceramic-based material, such as silica, in combination with a polymer, such as a water-soluble polymer, for example PVA. As noted above, examples of ceramic-based materials that can be used in the shell 46 include silica and bioactive glass. The polymer of the shell 46 can be the same as the polymer 48 or can comprise a different polymer. Examples of polymers that can be used in the shell 46 include, but are not limited to, PVA, PEO, PEG, gelatin, dextran, and carbohydrate.

Based on specific applications, the bioactive substance 44 can comprise bacteria 44, as depicted in FIG. 3, or can comprise another bioactive substance, such as drugs or other bioactive substances including but not limited to DNA, RNA, proteins, viruses, or mammalian cells.

FIG. 4 shows a cross-section of the core-shell fiber 40 through the bacteria 44 to show the polymer 48 of the core 42 encapsulating the bacteria 44, while the shell 46 encapsulates the core 42 of the polymer 48 and the bacteria 44. FIG. 5 shows a cross-section of the core-shell fiber 40 through a portion of the fiber 40 that does not include the bioactive substance 44, e.g., where only the polymer 48 and the shell 46 are present.

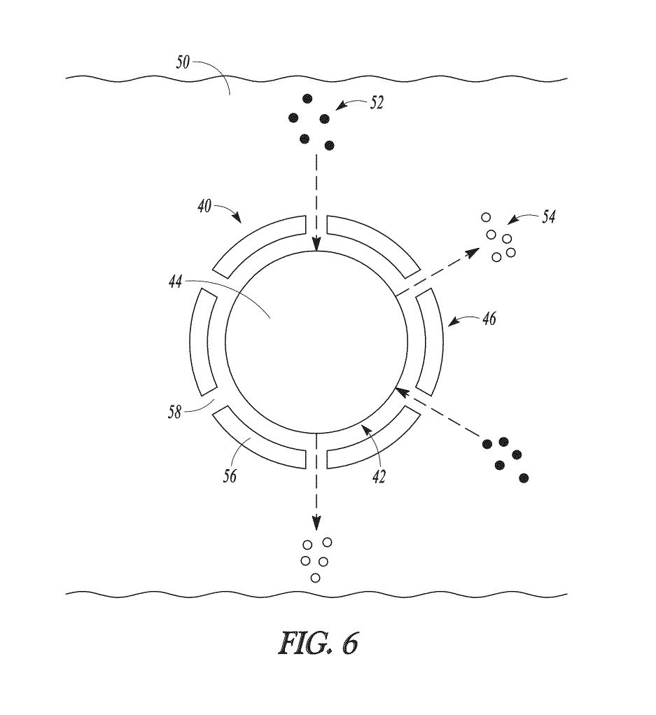

FIG. 6 shows a conceptual cross-sectional view of the example a bacteria-encapsulated core-shell fiber 40 located in an aqueous medium 50. For example, the aqueous medium 50 can comprise water that is contaminated with a contaminant 52, such as atrazine. The bacteria-encapsulated fiber 40 can be configured for bioremediation of the contaminant 52 from the aqueous medium 50. For example, the bacteria 44 can express an enzyme that can transform the contaminant 52 into a more desirable product 54, such as a non-toxic or substantially non-toxic or substantially non-harmful material. For example, in the case of atrazine bioremediation, the bacteria 44 can express atrazine chlorohydrolase that can transform atrazine, a regulated and toxic herbicide, into substantially non-toxic hydroxyatrazine.

As described above with respect to the system of FIGS. 1 and 2, the shell 46 of the fiber 40 can include a water-soluble polymer, such as PVA, PEG, and the like, that can act as a porogen when exposed to an aqueous environment. In an example, the aqueous medium 50 can dissolve the porogen polymer, which leaches out of the fiber shell 46 (FIGS. 3-5), leaving behind a non-soluble or substantially non-soluble ceramic (e.g., silica) fiber shell 56 with many pores 56 in the ceramic fiber shell 56. The pores 58 can further facilitate diffusion of the contaminants 52 in the aqueous medium 50 into the core 42 to reach the encapsulated bacteria 44. The bacteria 44, or enzymes expressed by the bacteria, can provide for biotransformation of the contaminants 52 into the non-toxic or substantially non-toxic products 54.

A microfluidic device, such as the microfluidic device 20 described above, coupled with a coaxial electrospinning system, such as system 10, can produce reactive mats comprising core-shell fibers, such as fiber 40, having a core 42 comprising a bioactive substance 44 and a ceramic-based shell 46 surrounding the core 42. An advantage of this technique over previous electrospinning methods is that ceramic-based fibers can be made continuously at low temperatures, such as room temperature, without using ethanol or any other organic solvents. Thus, the systems and method described herein can be capable of retaining the morphology of the fibers and the activity of the encapsulated bioactive substance even in an aqueous medium. Continuous formation of ceramic-based ultrafine fibers, e.g., silica-based fibers, with encapsulated bioactive substance was not attainable via previous electrospinning or any other existing techniques due to various limitations such as high temperature processes or organic solvents (such as ethanol) that would destroy or deactivate the bioactive substance, or a continuously changing viscosity of solution during electro spinning, which would result in the formation of droplets rather than continuously formed fibers.

### EXAMPLES

The embodiments of the present invention can be better understood by reference to the following examples which are offered by way of illustration. The present invention is not limited to the examples given herein.

### Example 1

### Preparation of Silica Precursor Solution and Thickener Solution

Tetramethyl orthosilicate (TMOS), hydrochloric acid (HCl), and polyvinyl alcohol (PVA) polymer (average molecular weight 50 kDa, 88% hydrolyzed) were obtained commercially from Sigma-Aldrich. Ultrapure water from a Millipore purification system was used throughout the present examples. All chemicals were used as received without further purification. PVA solutions were made by dissolving PVA in water at 80° C. while stirring. TMOS was hydrolyzed by sonication in the presence of HCl and the molar ratio of TMOS/water/HCl was 1:2.8:0.00024.

Fabrication of Silica/PVA Fibers by Conventional Electrospinning Coupled with a Microfluidic Device

Fibrous mats comprising silica and PVA composite fibers were fabricated via conventional electrospinning coupled with a microfluidic device, similar to the microfluidic device 20 described above. The hydrolyzed TMOS solution was loaded into a syringe connected to one inlet of a Y-shaped three-way adapter. The PVA solution at a concentration of 18% (w/v) was loaded into another syringe connected to the other inlet of the Y-shaped three-way adapter. Each syringe was attached to an individual syringe pump. The flow rates of the TMOS solution and the PVA solution were 11.4 μL/min and 8.6 μL/min, respectively, which ensured that the ratio of TMOS solution to PVA solution in the final solution is 4:3 by volume. The outlet of the three-way adapter was connected to an 18-gauge needle through the microfluidic device consisting of a silicone tubing. The length of the tubing was precisely controlled such that the reaction time (e.g., time taken for the mixture to reach the orifice of the needle from the mixing point through the microfluidic device) was 9 min. This was done to ensure that the solutions thoroughly mixed and the viscosity of the solution reached its optimum value for electro spinning.

The viscosity of the final solution was measured by a digital viscometer (NDJ-8S) and the measured viscosity was 4173±686 cP. A reaction time significantly shorter than 9 min resulted in formation of beaded fibers or droplets. A reaction time significantly longer than 9 min resulted in occurrence of gelation before the mixture reached the orifice of the needle and no fiber could be obtained.

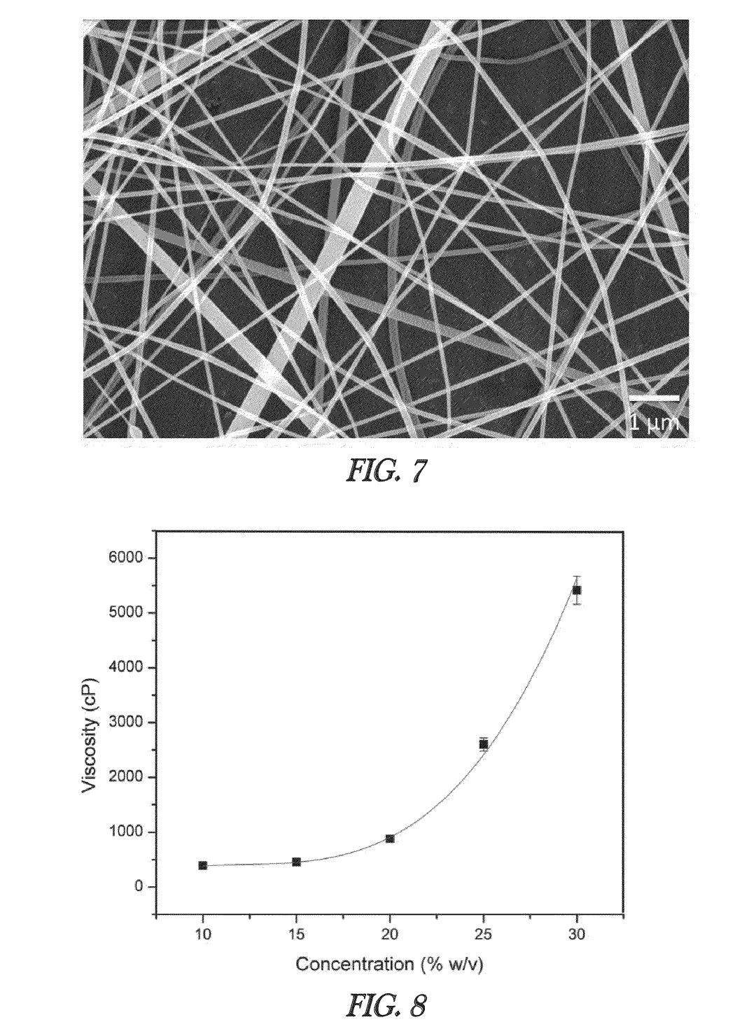

A voltage of 15 kV was applied to the needle by a high voltage power supply. A grounded conductive plate, which was placed 15 cm away from the tip of the needle, was used as the target to collect the spun fibers. Formation of uniform fibers without defects such as beads or droplets was verified by a scanning electron microscope (SEM) micrograph of the resulting fibrous mat (FIG. 7).

### Example 2

Fibrous mats comprising core-shell type fibers having a PVA core and a silica and PVA composite shell surrounding the core were fabricated via a coaxial electrospinning system coupled with a microfluidic device.

Optimization of the Viscosity of the Core Solution

The viscosity of the shell solution was optimized as described above with respect to the solution in Example 1. The viscosity of the core solution was optimized by measuring the viscosities of PVA solutions at different concentrations [10, 15, 20, 25, 30% (w/v)] (FIG. 8) and hence estimating the relationship between the viscosity of the PVA solution and the concentration of the PVA solution. A PVA concentration of 28% (w/v) corresponded to a solution viscosity of 4173 cP. So a PVA solution at a concentration of 28% (w/v) was used as the core solution in order to ensure that the core solution and the shell solution possessed similar viscosity, thus improving entrainment of the core solution and the shell solution.

Fabrication of Core-Shell Structured Fibers Having a PVA Core and a Silica and PVA Composite Shell Surrounding the Core

The core solution was loaded into a syringe that was connected to a 22-gauge needle (inner needle) placed concentrically inside a 18-gauge needle (outer needle). The shell solution was made by mixing a hydrolyzed TMOS solution with a PVA solution having a concentration of 18% (w/v). The hydrolyzed TMOS solution was loaded into a second syringe, which was connected to one inlet of the microfluidic device. The PVA solution was loaded into a third syringe, which was connected to the other inlet of the microfluidic device. The outlet of the microfluidic device was connected to the outer needle. The microfluidic device was used to precisely control the reaction of the hydrolyzed TMOS solution and the PVA solution such that the reaction time was 9 min. This was done to ensure that the solutions were thoroughly mixed and the viscosity of the solution reached its optimum value for electro spinning. Each syringe was attached to an individual syringe pump. The flow rates of the TMOS solution from the second syringe and the PVA solution from the third syringe were 11.4 μL/min and 8.6 μL/min, respectively, which ensured that the ratio of TMOS solution to PVA solution in the final shell solution is 4:3 by volume. The flow rate of the core solution from the first syringe was 10 μL/min.

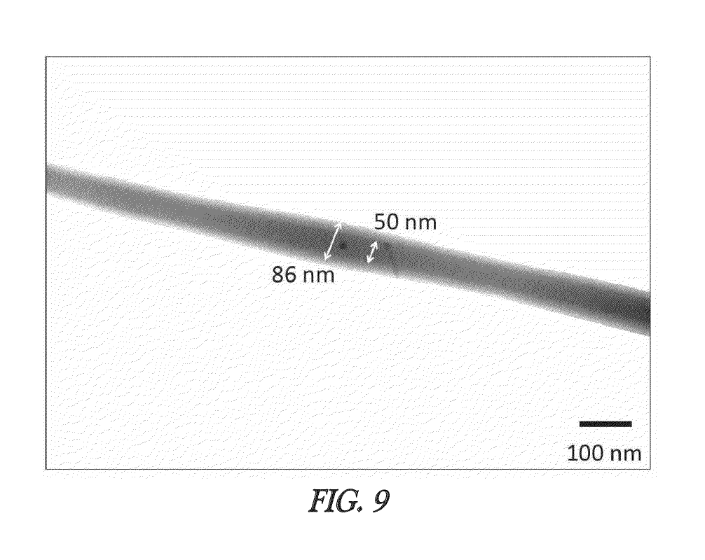

A voltage of 15 kV was applied to the outer needle by a high voltage power supply so that the shell solution was drawn by the electric force while the core solution was drawn by the shear force at the interface of the core solution and the shell solution. Reactive mats comprising core/shell structured nanofibers with a PVA core and a silica/PVA shell surrounding the core were collected by a grounded conductive plate, which was placed 15 cm away from the tip of the needle. Formation of core-shell type fibers was verified by a transmission electron microscope (TEM) image of the resulting fiber (FIG. 9).

### Example 3

### Preparation of Silica Precursor Solution and Thickener Solution

The silica precursor solution (e.g., TMOS solution) and thickener solution (e.g., PVA solution) were prepared as described in Example 1.

Fabrication of E. coli Bacteria Expressing GFP-Containing Silica/PVA Fibers by Conventional Electro Spinning Coupled with a Microfluidic Device

Fibrous mats comprising E. coli expressing GFP-containing silica and PVA composite fibers were fabricated via conventional electro spinning coupled with a microfluidic device. The hydrolyzed TMOS solution was loaded into a syringe connected to one inlet of a Y-shaped three-way adapter. E. coli expressing GFP pellets were mixed with the 18% (w/v) PVA solution at a cell density of 0.5 g/ml and the mixture was loaded into another syringe connected to the other inlet of the Y-shaped three-way adapter. Each syringe was attached to an individual syringe pump. The flow rates of the TMOS solution and the E. coli/PVA solution were 11.4 μL/min and 8.6 μL/min, respectively, which ensured that the ratio of TMOS solution to E. coli and PVA solution in the final solution is 4:3 by volume.

The outlet of the three-way adapter was connected to an 18-gauge needle through the microfluidic device consisting of a silicone tubing. The length of the tubing was precisely controlled such that the reaction time (e.g., time taken for the mixture to reach the orifice of the needle from the mixing point through the microfluidic device) was 9 min. Again, a reaction time significantly shorter than 9 min resulted in formation of beaded fibers or droplets whereas a reaction time significantly longer than 9 min resulted in occurrence of gelation before the mixture reached the orifice of the needle and no fiber could be obtained.

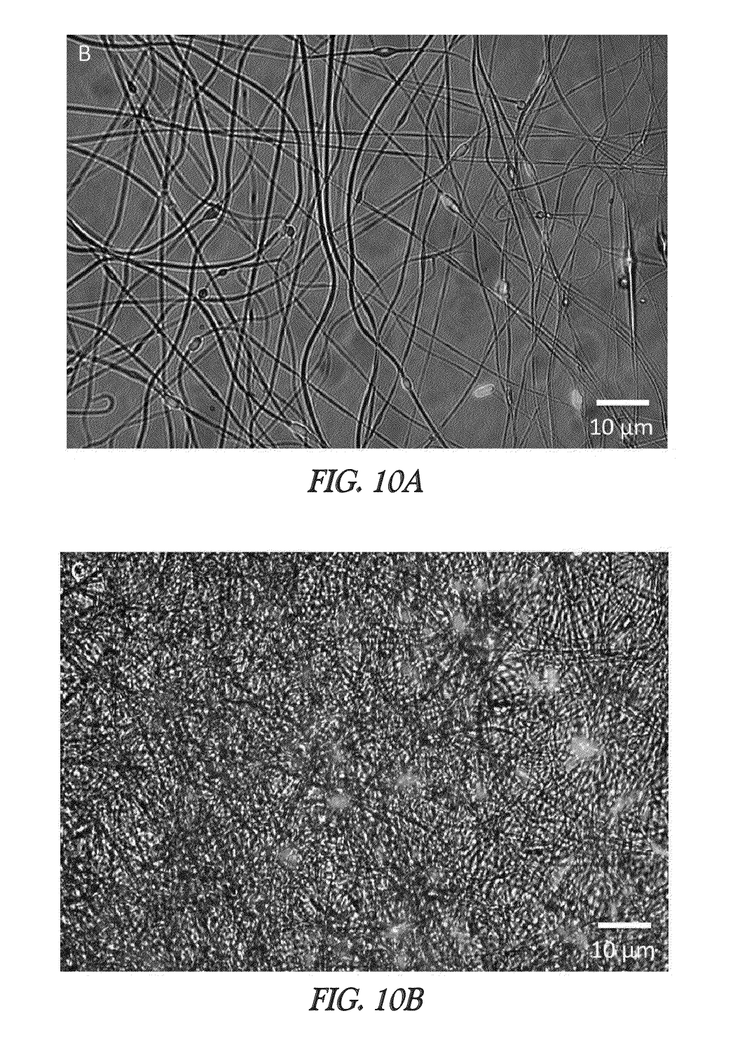

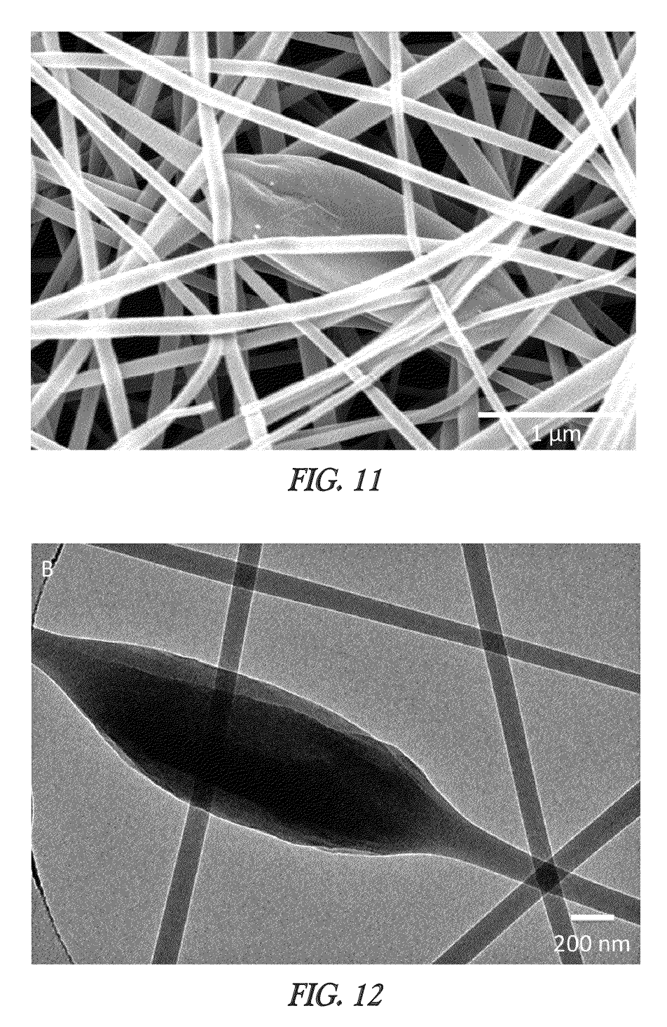

A voltage of 15 kV was applied to the needle by a high voltage power supply. A grounded conductive plate, which was placed 15 cm away from the tip of the needle, was used as the target to collect the spun fibers. Formation of E. coli expressing GFP-containing silica and PVA composite fibers was verified by fluorescence microscopy (FIGS. 10A and 10B), SEM (FIG. 11) and TEM (FIG. 12).

### Example 4

### Preparation of Silica Precursor Solution and Thickener Solution

The silica precursor solution (e.g., TMOS solution) and thickener solution (e.g., PVA solution) were prepared as described in Example 1.

Fabrication of Core-Shell Structured Fibers Having an E. coli Expressing GFP-Containing Core and a Silica-PVA Shell Surrounding the Core by Coaxial Electrospinning Coupled with a Microfluidic Device

To make a core solution, E. coli expressing GFP pellets were mixed with the PVA solution with a concentration of 28% (w/v) at a cell density of 0.5 g/ml and the mixture was loaded into a syringe (say, the first syringe), which was connected to a 22-gauge needle (inner needle) placed concentrically inside a 18-gauge needle (outer needle). A shell solution was made by mixing a hydrolyzed TMOS solution with the PVA solution having a concentration of 18% (w/v). The hydrolyzed TMOS solution was loaded into a second syringe, which was connected to one inlet of a microfluidic device. The 18% (w/v) PVA solution was loaded into a third syringe, which was connected to the other inlet of the microfluidic device. The outlet of the microfluidic device was connected to the outer needle. The microfluidic device was used to precisely control the reaction of the hydrolyzed TMOS solution and the PVA solution such that the reaction time was 9 min. This was done to ensure that the solutions were thoroughly mixed and the viscosity of the solution reached its optimum value for electro spinning. Each syringe was attached to an individual syringe pump. The flow rates of the TMOS solution from the second syringe and the PVA solution from the third syringe were 11.4 μL/min and 8.6 μL/min, respectively, which ensured that the ratio of TMOS solution to PVA solution in the final shell solution is 4:3 by volume.

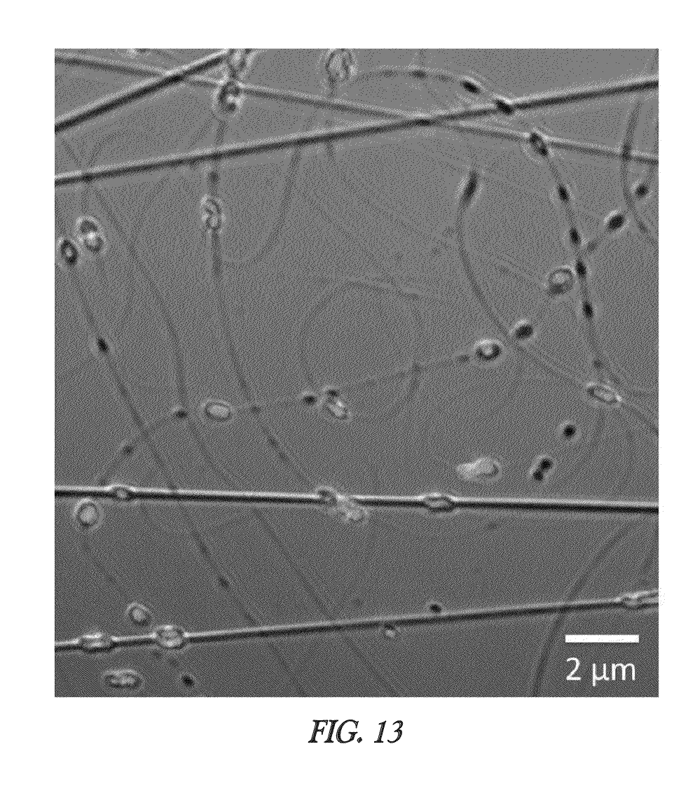

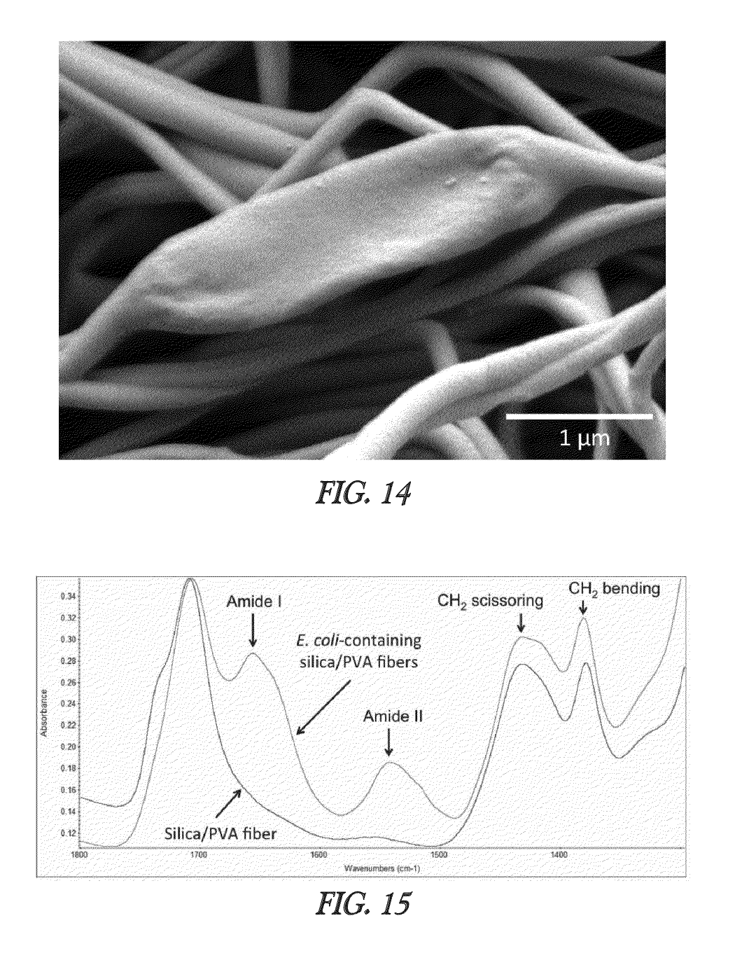

A voltage of 15 kV was applied to the outer needle by a high voltage power supply so that the shell solution was drawn by the electric force while the core solution was drawn by the shear force at the interface of the core solution and the shell solution. Reactive membranes comprising core/shell structured nanofibers with an E. coli expressing GFP-containing core and a silica and PVA composite shell surrounding the core were collected by a grounded conductive plate, which was placed 15 cm away from the tip of the needle. Formation of E. coli expressing GFP-containing silica and PVA composite fibers was verified by confocal microscopy (FIG. 13) and SEM (FIG. 14).

### Example 5

### Preparation of Silica Precursor Solution and Thickener Solution

The silica precursor solution (e.g., TMOS solution) and thickener solution (e.g., PVA solution) were prepared as described in Example 1.

Fabrication of E. coli Expressing AtzA-Containing Silica and PVA Composite Fibers by Conventional Electrospinning Coupled with a Microfluidic Device

Fibrous mats comprising E. coli expressing AtzA-containing silica and PVA composite fibers were fabricated via conventional electrospinning coupled with the microfluidic device. The fibrous mats were prepared as described in Example 3 except that the E. coli expressing GFP was replaced by the E. coli expressing AtzA.

Estimation of the Amount of E. coli in a Fibrous Mat

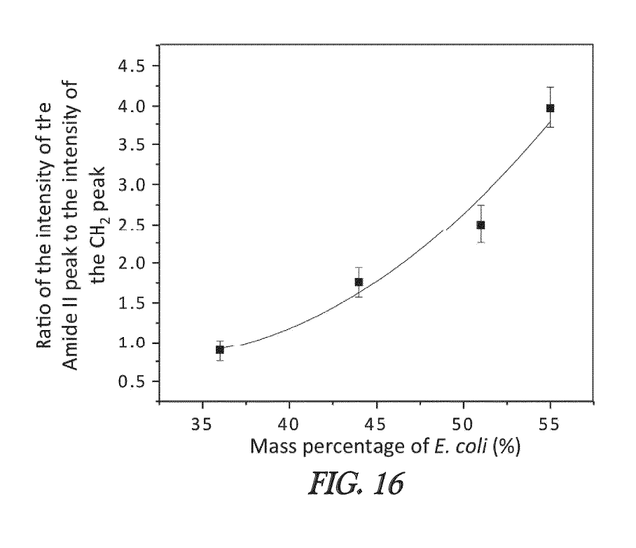

The amount of E. coli in the fibrous mat comprising E. coli-containing silica/PVA fibers was estimated by FTIR. FTIR analysis was conducted for silica and PVA composite fibers without E. coli, E. coli-containing silica and PVA composite fibers, and silica and PVA with known percentages of E. coli, using a Nicolet Continuum FTIR microscope. IR spectra in the range of 400-4000 cm−1 were recorded at a resolution of 4 cm−1 (FIG. 15). Each sample was scanned at five different regions with an aperture size of 100×100 μm. Calculation of the amount of the E. coli in the silica and PVA composite fibrous mat was based on the ratio of the intensity of the Amide II peak (corresponding to cellular proteins) to the intensity of the methylene peak (originating from silica and PVA) (FIG. 16). The results showed that the mass percentage of E. coli in the electro spun silica/PVA fibrous mat was 44% g/g.

Atrazine Bioremediation by Reactive Mats Comprising E. coli Expressing AtzA-Containing Silica/PVA Fibers Fabricated Via Conventional Electrospinning Coupled with a Microfluidic Device

Reactive fibrous mats with AtzA E. coli were weighed directly after electro spinning. The mass of the bacteria was estimated by comparing the FTIR spectra of the E. coli-containing silica and PVA composite fibers with those of the silica and PVA mixture containing a known mass of E. coli. Activity measurements of the reactive mats comprising E. coli expressing AtzA-containing silica and PVA composite fibers were conducted at room temperature in 20 ml glass scintillation vials. The reaction was initiated by exposing the bacterium-containing fibers to 5 ml of 0.1 M potassium phosphate buffer (at pH 7.0) containing 150 μM (32.4 ppm) atrazine. The solution was stirred continuously using an orbital shaker. The supernatant was sampled after 20 min and the sample was then filtered through a 0.2 μm pore size PTFE syringe filter to filter out any silica fragments or bacteria that may have been released. The sample was heated to boiling point for 10 min to ensure that any released enzyme was denatured. In the sample solution, the concentrations of atrazine and its metabolite, hydroxyatrazine, were measured by high-performance liquid chromatography (HPLC).

Activity measurements of the free bacteria were conducted in a similar fashion. The specific activities of the encapsulated bacteria were expressed in two ways, based on μmol of atrazine degraded (or hydroxyatrazine produced) per gram of E. coli per minute (μmol/g of E. coli/min) and μmol of atrazine degraded (or hydroxyatrazine produced) per gram of nanofibers per minute (μmol/g of nanofibers/min). The specific activities of the free bacteria were expressed as μmol of atrazine degraded (or hydroxyatrazine produced) per gram of E. coli per minute (μmol/g of E. coli/min). The results showed that the atrazine biodegradation rate of the bacterium-containing fibers was 0.25 μmol/g of fibers/min or 0.57 μmol/g of E. coli/min, which was comparable to that of the free bacteria (0.64 μmol/g of E. coli/min). The rate of atrazine degraded or adsorbed was comparable to that of hydroxyatrazine produced, implying that adsorption of atrazine into silica was negligible.

### Example 6

### Preparation of Silica Precursor Solution and Thickener Solution

The silica precursor solution (e.g., TMOS solution) and thickener solution (e.g., PVA solution) were prepared as described in Example 1.

Fabrication of Core/Shell Structured Fibers Having an E. coli Expressing AtzA-Containing Core and a Silica/PVA Shell Surrounding the Core by Coaxial Electrospinning Coupled with a Microfluidic Device

Reactive mats comprising core-shell fibers having an E. coli expressing AtzA-containing core and a silica and PVA composite shell surrounding the core were made as described in Example 4 except that the E. coli expressing GFP was replaced by the E. coli expressing AtzA.

Estimation of the Amount of E. coli in a Fibrous Mat

The amount of E. coli in the fibrous mat comprising core/shell structured fibers with an E. coli expressing AtzA core and a silica/PVA shell was estimated by FTIR as described in Example 5. The results showed that the mass percentage of E. coli in the electro spun silica/PVA fibrous mat was 40% g/g.

Atrazine Bioremediation by Reactive Mats Comprising Core-Shell Fibers Having an E. coli Expressing AtzA-Containing Core and a Silica and PVA Composite Shell Surrounding the Core by Coaxial Electrospinning Coupled with a Microfluidic Device

Atrazine bioremediation by the reactive mats was assayed as described in Example 5. The results showed that the atrazine biodegradation rate of the bacterium-containing core-shell type fibers was 0.10 μmol/g of fibers/min or 0.24 μmol/g of E. coli/min. Again, the rate of atrazine degraded or adsorbed was comparable to that of hydroxyatrazine produced, implying that adsorption of atrazine into silica was negligible.

To better illustrate the concepts of the present disclosure, a non-limiting list of EMBODIMENTS is provided here:

EMBODIMENT 1 can include subject matter (such as an apparatus, a device, a method, or one or more means for performing acts), such as can include an ultrafine fiber comprising a ceramic-based fibrous body and a biologically active substance encapsulated in the body, substantially encapsulated in the body, or surface-attached to the body.

EMBODIMENT 2 can include, or can optionally be combined with the subject matter of EMBODIMENT 1, to optionally include the body comprising a polymer and a ceramic gel network.

EMBODIMENT 3 can include, or can optionally be combined with the subject matter of EMBODIMENT 2, to optionally include the polymer comprising a water-soluble polymer.

EMBODIMENT 4 can include, or can optionally be combined with the subject matter of one or any combination of EMBODIMENTS 2 and 3, to optionally include the polymer comprising polyvinyl alcohol.

EMBODIMENT 5 can include, or can optionally be combined with the subject matter of one or any combination of EMBODIMENTS 2-4, to optionally include the polymer comprising polyethylene oxide.

EMBODIMENT 6 can include, or can optionally be combined with the subject matter of one or any combination of EMBODIMENTS 2-5, to optionally include the polymer comprising polyethylene glycol.

EMBODIMENT 7 can include, or can optionally be combined with the subject matter of one or any combination of EMBODIMENTS 2-6, to optionally include the polymer comprising gelatin.

EMBODIMENT 8 can include, or can optionally be combined with the subject matter of one or any combination of EMBODIMENTS 2-7, to optionally include the polymer comprising dextran.

EMBODIMENT 9 can include, or can optionally be combined with the subject matter of one or any combination of EMBODIMENTS 2-8, to optionally include the polymer comprising a carbohydrate.

EMBODIMENT 10 can include, or can optionally be combined with the subject matter of one or any combination of EMBODIMENTS 2-9, to optionally include the polymer comprising at least one of polyvinyl alcohol, polyethylene oxide, polyethylene glycol, gelatin, dextran, and a carbohydrate.

EMBODIMENT 11 can include, or can optionally be combined with the subject matter of one or any combination of EMBODIMENTS 2-10, to optionally include the ceramic gel network comprising a silica gel network.

EMBODIMENT 12 can include, or can optionally be combined with the subject matter of one or any combination of EMBODIMENTS 2-11, to optionally include the ceramic gel network comprising a bioactive glass gel network.

EMBODIMENT 13 can include, or can optionally be combined with the subject matter of one or any combination of EMBODIMENTS 2-12, to optionally include the ceramic gel network comprising a silica gel network and a bioactive glass gel network.

EMBODIMENT 14 can include, or can optionally be combined with the subject matter of one or any combination of EMBODIMENTS 1-13, to optionally include the biologically active substance comprising deoxyribonucleic acid (DNA).

EMBODIMENT 15 can include, or can optionally be combined with the subject matter of one or any combination of EMBODIMENTS 1-14, to optionally include the biologically active substance comprising ribonucleic acid (RNA).

EMBODIMENT 16 can include, or can optionally be combined with the subject matter of one or any combination of EMBODIMENTS 1-15, to optionally include the biologically active substance comprising one or more proteins.

EMBODIMENT 17 can include, or can optionally be combined with the subject matter of one or any combination of EMBODIMENTS 1-16, to optionally include the biologically active substance comprising one or more viruses.

EMBODIMENT 18 can include, or can optionally be combined with the subject matter of one or any combination of EMBODIMENTS 1-17, to optionally include the biologically active substance comprising one or more bacteria.

EMBODIMENT 19 can include, or can optionally be combined with the subject matter of one or any combination of EMBODIMENTS 1-18, to optionally include the biologically active substance comprising one or more types of mammalian cells.

EMBODIMENT 20 can include, or can optionally be combined with the subject matter of one or any combination of EMBODIMENTS 1-19, to optionally include the biologically active substance comprising one or more of DNA, RNA, one or more proteins, one or more viruses, bacteria, and mammalian cells.

EMBODIMENT 21 can include, or can optionally be combined with the subject matter of one or any combination of EMBODIMENTS 1-20, to optionally include the fibrous body comprising a nanofiber.

EMBODIMENT 22 can include, or can optionally be combined with the subject matter of one or any combination of EMBODIMENTS 1-21, to optionally include the fibrous body comprising a microfiber.

EMBODIMENT 23 can include, or can optionally be combined with the subject matter of one or any combination of EMBODIMENTS 1-22, to optionally include the fibrous body being a nanofiber or a microfiber.

EMBODIMENT 24 can include, or can optionally be combined with the subject matter of one or any combination of EMBODIMENTS 1-23, to include subject matter (such as an apparatus, a device, a method, or one or more means for performing acts), such as can include an ultrafine fiber comprising a core comprising a biologically active substance and a ceramic-based shell surrounding or substantially surrounding the core.

EMBODIMENT 25 can include, or can optionally be combined with the subject matter of EMBODIMENT 24, to optionally include the body comprising a core polymer encapsulating or substantially encapsulating the biologically active substance.

EMBODIMENT 26 can include, or can optionally be combined with the subject matter of EMBODIMENT 25, to optionally include the core polymer comprising a water-soluble polymer.

EMBODIMENT 27 can include, or can optionally be combined with the subject matter of one or any combination of EMBODIMENTS 25 and 26, to optionally include the core polymer comprising polyvinyl alcohol.

EMBODIMENT 28 can include, or can optionally be combined with the subject matter of one or any combination of EMBODIMENTS 25-27, to optionally include the core polymer comprising polyethylene oxide.

EMBODIMENT 29 can include, or can optionally be combined with the subject matter of one or any combination of EMBODIMENTS 25-28, to optionally include the core polymer comprising polyethylene glycol.

EMBODIMENT 30 can include, or can optionally be combined with the subject matter of one or any combination of EMBODIMENTS 25-29, to optionally include the core polymer comprising gelatin.

EMBODIMENT 31 can include, or can optionally be combined with the subject matter of one or any combination of EMBODIMENTS 25-32, to optionally include the core polymer comprising dextran.

EMBODIMENT 32 can include, or can optionally be combined with the subject matter of one or any combination of EMBODIMENTS 25-31, to optionally include the core polymer comprising a carbohydrate.

EMBODIMENT 33 can include, or can optionally be combined with the subject matter of one or any combination of EMBODIMENTS 25-32, to optionally include the core polymer comprising one or more of polyvinyl alcohol, polyethylene oxide, polyethylene glycol, gelatin, dextran, and a carbohydrate.

EMBODIMENT 34 can include, or can optionally be combined with the subject matter of one or any combination of EMBODIMENTS 24-33, to optionally include the biologically active substance comprising deoxyribonucleic acid (DNA).

EMBODIMENT 35 can include, or can optionally be combined with the subject matter of one or any combination of EMBODIMENTS 24-34, to optionally include the biologically active substance comprising ribonucleic acid (RNA).

EMBODIMENT 36 can include, or can optionally be combined with the subject matter of one or any combination of EMBODIMENTS 24-35, to optionally include the biologically active substance comprising one or more proteins.

EMBODIMENT 37 can include, or can optionally be combined with the subject matter of one or any combination of EMBODIMENTS 24-36, to optionally include the biologically active substance comprising one or more viruses.

EMBODIMENT 38 can include, or can optionally be combined with the subject matter of one or any combination of EMBODIMENTS 24-37, to optionally include the biologically active substance comprising one or more bacteria.

EMBODIMENT 39 can include, or can optionally be combined with the subject matter of one or any combination of EMBODIMENTS 24-38, to optionally include the biologically active substance comprising one or more types of mammalian cells.

EMBODIMENT 40 can include, or can optionally be combined with the subject matter of one or any combination of EMBODIMENTS 24-39, to optionally include the biologically active substance comprising one or more of DNA, RNA, one or more proteins, one or more viruses, bacteria, and mammalian cells.

EMBODIMENT 41 can include, or can optionally be combined with the subject matter of one or any combination of EMBODIMENTS 24-40, to optionally include the shell comprising a shell polymer and a ceramic gel network.

EMBODIMENT 42 can include, or can optionally be combined with the subject matter of EMBODIMENT 41, to optionally include the shell polymer comprising a water-soluble polymer.

EMBODIMENT 43 can include, or can optionally be combined with the subject matter of one or any combination of EMBODIMENTS 41 and 42, to optionally include the shell polymer comprising polyvinyl alcohol.

EMBODIMENT 44 can include, or can optionally be combined with the subject matter of one or any combination of EMBODIMENTS 41-43, to optionally include the shell polymer comprising polyethylene oxide.

EMBODIMENT 45 can include, or can optionally be combined with the subject matter of one or any combination of EMBODIMENTS 41-44, to optionally include the shell polymer comprising polyethylene glycol.

EMBODIMENT 46 can include, or can optionally be combined with the subject matter of one or any combination of EMBODIMENTS 41-45, to optionally include the shell polymer comprising gelatin.

EMBODIMENT 47 can include, or can optionally be combined with the subject matter of one or any combination of EMBODIMENTS 41-46, to optionally include the shell polymer comprising dextran.

EMBODIMENT 48 can include, or can optionally be combined with the subject matter of one or any combination of EMBODIMENTS 41-47, to optionally include the shell polymer comprising a carbohydrate.

EMBODIMENT 49 can include, or can optionally be combined with the subject matter of one or any combination of EMBODIMENTS 41-48, to optionally include the shell polymer comprising one or more of polyvinyl alcohol, polyethylene oxide, polyethylene glycol, gelatin, dextran, and a carbohydrate.

EMBODIMENT 50 can include, or can optionally be combined with the subject matter of EMBODIMENT 41, to optionally include the ceramic gel network comprising a silica gel network.

EMBODIMENT 51 can include, or can optionally be combined with the subject matter of EMBODIMENT 41, to optionally include the ceramic gel network comprising a bio active glass gel network.

EMBODIMENT 52 can include, or can optionally be combined with the subject matter of EMBODIMENT 41, to optionally include the ceramic gel network comprising a silica gel network and a bioactive glass gel network.

EMBODIMENT 53 can include, or can optionally be combined with the subject matter of one or any combination of EMBODIMENTS 24-52, to optionally include the core and the shell forming a nanofiber.

EMBODIMENT 54 can include, or can optionally be combined with the subject matter of one or any combination of EMBODIMENTS 24-53, to optionally include the core and the shell forming a microfiber.

EMBODIMENT 55 can include, or can optionally be combined with the subject matter of one or any combination of EMBODIMENTS 24-54, to optionally include the core and the shell forming a nanofiber or a microfiber.

EMBODIMENT 56 can include, or can optionally be combined with the subject matter of one or any combination of EMBODIMENTS 1-54, to include subject matter (such as an apparatus, a device, a method, or one or more means for performing acts), such as can include a reactive mat comprising a plurality of ultrafine fibers, each ultrafine fiber comprising a ceramic-based fibrous body, and a biologically active substance encapsulated in the body, substantially encapsulated in the body, or surface-attached to the body.

EMBODIMENT 57 can include, or can optionally be combined with the subject matter of EMBODIMENT 56, to optionally include the body of each ultrafine fiber comprising a polymer and a ceramic gel network.

EMBODIMENT 58 can include, or can optionally be combined with the subject matter of EMBODIMENT 57, to optionally include the polymer comprising a water-soluble polymer.

EMBODIMENT 59 can include, or can optionally be combined with the subject matter of one or any combination of EMBODIMENTS 57 and 58, to optionally include the polymer comprising polyvinyl alcohol.

EMBODIMENT 60 can include, or can optionally be combined with the subject matter of one or any combination of EMBODIMENTS 57-59, to optionally include the polymer comprising polyethylene oxide.

EMBODIMENT 61 can include, or can optionally be combined with the subject matter of one or any combination of EMBODIMENTS 57-60, to optionally include the polymer comprising polyethylene glycol.

EMBODIMENT 62 can include, or can optionally be combined with the subject matter of one or any combination of EMBODIMENTS 57-61, to optionally include the polymer comprising gelatin.

EMBODIMENT 63 can include, or can optionally be combined with the subject matter of one or any combination of EMBODIMENTS 57-62, to optionally include the polymer comprising dextran.

EMBODIMENT 64 can include, or can optionally be combined with the subject matter of one or any combination of EMBODIMENTS 57-63, to optionally include the polymer comprising a carbohydrate.

EMBODIMENT 65 can include, or can optionally be combined with the subject matter of one or any combination of EMBODIMENTS 57-64, to optionally include the polymer comprising one or more of polyvinyl alcohol, polyethylene oxide, polyethylene glycol, gelatin, dextran, and a carbohydrate.

EMBODIMENT 66 can include, or can optionally be combined with the subject matter of EMBODIMENT 57, to optionally include the ceramic gel network comprising a silica gel network.

EMBODIMENT 67 can include, or can optionally be combined with the subject matter of EMBODIMENT 57, to optionally include the ceramic gel network comprising a bio active glass gel network.

EMBODIMENT 68 can include, or can optionally be combined with the subject matter of EMBODIMENT 57, to optionally include the ceramic gel network comprising a silica gel network and a bioactive glass gel network.

EMBODIMENT 69 can include, or can optionally be combined with the subject matter of one or any combination of EMBODIMENTS 56-68, to optionally include the biologically active substance comprising deoxyribonucleic acid (DNA).

EMBODIMENT 70 can include, or can optionally be combined with the subject matter of one or any combination of EMBODIMENTS 56-69, to optionally include the biologically active substance comprising ribonucleic acid (RNA).

EMBODIMENT 71 can include, or can optionally be combined with the subject matter of one or any combination of EMBODIMENTS 56-70, to optionally include the biologically active substance comprising one or more proteins.

EMBODIMENT 72 can include, or can optionally be combined with the subject matter of one or any combination of EMBODIMENTS 56-71, to optionally include the biologically active substance comprising one or more viruses.

EMBODIMENT 73 can include, or can optionally be combined with the subject matter of one or any combination of EMBODIMENTS 56-72, to optionally include the biologically active substance comprising one or more bacteria.

EMBODIMENT 74 can include, or can optionally be combined with the subject matter of one or any combination of EMBODIMENTS 56-73, to optionally include the biologically active substance comprising one or more types of mammalian cells.

EMBODIMENT 75 can include, or can optionally be combined with the subject matter of one or any combination of EMBODIMENTS 56-74, to optionally include the biologically active substance comprising one or more of DNA, RNA, one or more proteins, one or more viruses, bacteria, and mammalian cells.

EMBODIMENT 76 can include, or can optionally be combined with the subject matter of one or any combination of EMBODIMENTS 56-75, to optionally include the fibrous body of each ultrafine fiber being a nanofiber or a microfiber.

EMBODIMENT 77 can include, or can optionally be combined with the subject matter of one or any combination of EMBODIMENTS 1-76, to include subject matter (such as an apparatus, a device, a method, or one or more means for performing acts), such as can include a reactive mat comprising a plurality of ultrafine fibers, each ultrafine fiber comprising a core comprising a biologically active substance, and a ceramic-based shell surrounding or substantially surrounding the core.

EMBODIMENT 78 can include, or can optionally be combined with the subject matter of EMBODIMENT 77, to optionally include the core of each ultrafine fiber comprising a core polymer encapsulating or substantially encapsulating the biologically active substance.

EMBODIMENT 79 can include, or can optionally be combined with the subject matter of EMBODIMENTS 78, to optionally include the core polymer comprising a water-soluble polymer.

EMBODIMENT 80 can include, or can optionally be combined with the subject matter of EMBODIMENT 78 and 79, to optionally include the core polymer comprising polyvinyl alcohol.

EMBODIMENT 81 can include, or can optionally be combined with the subject matter of one or any combination of EMBODIMENTS 78-80, to optionally include the core polymer comprising polyethylene oxide.

EMBODIMENT 82 can include, or can optionally be combined with the subject matter of one or any combination of EMBODIMENTS 78-81, to optionally include the core polymer comprising polyethylene glycol.

EMBODIMENT 83 can include, or can optionally be combined with the subject matter of one or any combination of EMBODIMENTS 78-82, to optionally include the core polymer comprising gelatin.

EMBODIMENT 84 can include, or can optionally be combined with the subject matter of one or any combination of EMBODIMENTS 78-83, to optionally include the core polymer comprising dextran.

EMBODIMENT 85 can include, or can optionally be combined with the subject matter of one or any combination of EMBODIMENTS 78-84, to optionally include the core polymer comprising a carbohydrate.

EMBODIMENT 86 can include, or can optionally be combined with the subject matter of one or any combination of EMBODIMENTS 78-85, to optionally include the core polymer comprising one or more of polyvinyl alcohol, polyethylene oxide, polyethylene glycol, gelatin, dextran, and a carbohydrate.

EMBODIMENT 87 can include, or can optionally be combined with the subject matter of one or any combination of EMBODIMENTS 77-86, to optionally include the biologically active substance comprising deoxyribonucleic acid (DNA).

EMBODIMENT 88 can include, or can optionally be combined with the subject matter of one or any combination of EMBODIMENTS 77-87, to optionally include the biologically active substance comprising ribonucleic acid (RNA).

EMBODIMENT 89 can include, or can optionally be combined with the subject matter of one or any combination of EMBODIMENTS 77-88, to optionally include the biologically active substance comprising one or more proteins.

EMBODIMENT 90 can include, or can optionally be combined with the subject matter of one or any combination of EMBODIMENTS 77-89, to optionally include the biologically active substance comprising one or more viruses.

EMBODIMENT 91 can include, or can optionally be combined with the subject matter of one or any combination of EMBODIMENTS 77-90, to optionally include the biologically active substance comprising one or more bacteria.

EMBODIMENT 92 can include, or can optionally be combined with the subject matter of one or any combination of EMBODIMENTS 77-91, to optionally include the biologically active substance comprising one or more types of mammalian cells.

EMBODIMENT 93 can include, or can optionally be combined with the subject matter of one or any combination of EMBODIMENTS 77-92, to optionally include the biologically active substance comprising one or more of DNA, RNA, one or more proteins, one or more viruses, bacteria, and mammalian cells.

EMBODIMENT 94 can include, or can optionally be combined with the subject matter of one or any combination of EMBODIMENTS 77-93, to optionally include the shell of each ultrafine fiber comprising a shell polymer and a ceramic gel network.

EMBODIMENT 95 can include, or can optionally be combined with the subject matter of EMBODIMENT 94, to optionally include the shell polymer comprising a water-soluble polymer.

EMBODIMENT 96 can include, or can optionally be combined with the subject matter of one or any combination of EMBODIMENTS 94 and 95, to optionally include the shell polymer comprising polyvinyl alcohol.

EMBODIMENT 97 can include, or can optionally be combined with the subject matter of one or any combination of EMBODIMENTS 94-96, to optionally include the shell polymer comprising polyethylene oxide.

EMBODIMENT 98 can include, or can optionally be combined with the subject matter of one or any combination of EMBODIMENTS 94-97, to optionally include the shell polymer comprising polyethylene glycol.

EMBODIMENT 99 can include, or can optionally be combined with the subject matter of one or any combination of EMBODIMENTS 94-98, to optionally include the shell polymer comprising gelatin.

EMBODIMENT 100 can include, or can optionally be combined with the subject matter of one or any combination of EMBODIMENTS 94-99, to optionally include the shell polymer comprising dextran.

EMBODIMENT 101 can include, or can optionally be combined with the subject matter of one or any combination of EMBODIMENTS 94-100, to optionally include the shell polymer comprising a carbohydrate.

EMBODIMENT 102 can include, or can optionally be combined with the subject matter of one or any combination of EMBODIMENTS 94-100, to optionally include the shell polymer comprising one or more of polyvinyl alcohol, polyethylene oxide, polyethylene glycol, gelatin, dextran, and a carbohydrate.

EMBODIMENT 103 can include, or can optionally be combined with the subject matter of one or any combination of EMBODIMENTS 94-102, to optionally include the ceramic gel network comprising a silica gel network.

EMBODIMENT 104 can include, or can optionally be combined with the subject matter of one or any combination of EMBODIMENTS 94-103, to optionally include the ceramic gel network comprising a bioactive glass gel network.

EMBODIMENT 105 can include, or can optionally be combined with the subject matter of one or any combination of EMBODIMENTS 94-104, to optionally include the ceramic gel network comprising a silica gel network and a bioactive glass gel network.

EMBODIMENT 106 can include, or can optionally be combined with the subject matter of one or any combination of EMBODIMENTS 77-105, to optionally include the core and the shell of each ultrafine fiber forming a nanofiber or a microfiber.

EMBODIMENT 107 can include, or can optionally be combined with the subject matter of one or any combination of EMBODIMENTS 1-106, to include subject matter (such as an apparatus, a device, a method, or one or more means for performing acts), such as can include a system comprising a ceramic precursor supply system configured to supply a ceramic precursor solution, a polymer supply system configured to supply a polymer solution, a microfluidic device configured to mix and react the ceramic precursor solution and the polymer solution to form an electro spinning solution having a predetermined viscosity, and an electrospinning device configured to electrospin the electrospinning solution to form a fibrous body comprising a ceramic formed from the ceramic precursor solution and a polymer formed from the polymer solution.

EMBODIMENT 108 can include, or can optionally be combined with the subject matter of EMBODIMENT 107, to optionally include the microfluidic device comprising an adapter configured to receive the ceramic precursor from the ceramic precursor supply system and the polymer from the polymer supply system and to combine the ceramic precursor and the polymer.

EMBODIMENT 109 can include, or can optionally be combined with the subject matter of one or any combination of EMBODIMENTS 107 and 108, to optionally include the microfluidic device comprising a tubing configured to provide for a predetermined residence time of the ceramic precursor and the polymer precursor within the tubing.

EMBODIMENT 110 can include, or can optionally be combined with the subject matter of one or any combination of EMBODIMENTS 107-109, to optionally include the electrospinning device comprising an electrospinning conduit configured to receive the electrospinning solution, a power supply configured to apply a voltage to the conduit, and a conductive plate to collect the fibrous body.

EMBODIMENT 111 can include, or can optionally be combined with the subject matter of one or any combination of EMBODIMENTS 107-110, to optionally include the electrospinning device comprising an inner conduit nested within the electrospinning conduit.

EMBODIMENT 112 can include, or can optionally be combined with the subject matter of one or any combination of EMBODIMENTS 107-111, to optionally include the system further comprising a core solution supply system configured to supply a core solution to the inner conduit.

EMBODIMENT 113 can include, or can optionally be combined with the subject matter of one or any combination of EMBODIMENTS 107-112, to optionally include the electrospinning device being configured to form a core-shell type fiber comprising a shell of the ceramic and the polymer and a core surrounded or substantially surrounded by the shell.

EMBODIMENT 114 can include, or can optionally be combined with the subject matter of one or any combination of EMBODIMENTS 107-113, to optionally include the core solution comprising a core polymer solution and a biologically active substance dispersed in the core polymer solution.

EMBODIMENT 115 can include, or can optionally be combined with the subject matter of EMBODIMENT 114, to optionally include the core polymer solution comprising a solution of polyvinyl alcohol.

EMBODIMENT 116 can include, or can optionally be combined with the subject matter of one or any combination of EMBODIMENTS 114 AND 115, to optionally include the core polymer solution comprising a solution of polyethylene oxide.

EMBODIMENT 117 can include, or can optionally be combined with the subject matter of one or any combination of EMBODIMENTS 114-116, to optionally include the core polymer solution comprising a solution of polyethylene glycol.

EMBODIMENT 118 can include, or can optionally be combined with the subject matter of one or any combination of EMBODIMENTS 114-117, to optionally include the core polymer solution comprising a solution of gelatin.

EMBODIMENT 119 can include, or can optionally be combined with the subject matter of one or any combination of EMBODIMENTS 114-118, to optionally include the core polymer solution comprising a solution of dextran.

EMBODIMENT 120 can include, or can optionally be combined with the subject matter of one or any combination of EMBODIMENTS 114-119, to optionally include the core polymer solution comprising a solution of a carbohydrate.

EMBODIMENT 121 can include, or can optionally be combined with the subject matter of one or any combination of EMBODIMENTS 114-120, to optionally include the core polymer solution comprising a solution of one or more of polyvinyl alcohol, polyethylene oxide, polyethylene glycol, gelatin, dextran, and carbohydrate.

EMBODIMENT 122 can include, or can optionally be combined with the subject matter of EMBODIMENT 114, to optionally include the biologically active substance comprising deoxyribonucleic acid (DNA).

EMBODIMENT 123 can include, or can optionally be combined with the subject matter of one or any combination of EMBODIMENTS 114 and 122, to optionally include the biologically active substance comprising ribonucleic acid (RNA).

EMBODIMENT 124 can include, or can optionally be combined with the subject matter of one or any combination of EMBODIMENTS 114, 123, and 124, to optionally include the biologically active substance comprising one or more proteins.

EMBODIMENT 125 can include, or can optionally be combined with the subject matter of one or any combination of EMBODIMENTS 114 and 123-124, to optionally include the biologically active substance comprising one or more viruses.

EMBODIMENT 126 can include, or can optionally be combined with the subject matter of one or any combination of EMBODIMENTS 114 and 123-125, to optionally include the biologically active substance comprising one or more bacteria.

EMBODIMENT 127 can include, or can optionally be combined with the subject matter of one or any combination of EMBODIMENTS 114 and 123-126, to optionally include the biologically active substance comprising one or more types of mammalian cells.

EMBODIMENT 128 can include, or can optionally be combined with the subject matter of one or any combination of EMBODIMENTS 114 and 123-127, to optionally include the biologically active substance comprising one or more of DNA, RNA, one or more proteins, one or more viruses, bacteria, and mammalian cells.

EMBODIMENT 129 can include, or can optionally be combined with the subject matter of one or any combination of EMBODIMENTS 1-126, to optionally include the polymer solution comprising a water-soluble polymer.

EMBODIMENT 130 can include, or can optionally be combined with the subject matter of one or any combination of EMBODIMENTS 107-129, to optionally include the polymer solution comprising a solution of polyvinyl alcohol.

EMBODIMENT 131 can include, or can optionally be combined with the subject matter of one or any combination of EMBODIMENTS 107-130, to optionally include the polymer solution comprising a solution of polyethylene oxide.

EMBODIMENT 132 can include, or can optionally be combined with the subject matter of one or any combination of EMBODIMENTS 107-131, to optionally include the polymer solution comprising a solution of polyethylene glycol.

EMBODIMENT 133 can include, or can optionally be combined with the subject matter of one or any combination of EMBODIMENTS 107-132, to optionally include the polymer solution comprising a solution of gelatin.

EMBODIMENT 134 can include, or can optionally be combined with the subject matter of one or any combination of EMBODIMENTS 107-133, to optionally include the polymer solution comprising a solution of dextran.

EMBODIMENT 135 can include, or can optionally be combined with the subject matter of one or any combination of EMBODIMENTS 107-134, to optionally include the polymer solution comprising a solution of a carbohydrate.

EMBODIMENT 136 can include, or can optionally be combined with the subject matter of one or any combination of EMBODIMENTS 107-135, to optionally include the polymer solution comprising a solution of one or more of polyvinyl alcohol, polyethylene oxide, polyethylene glycol, gelatin, dextran, and carbohydrate.

EMBODIMENT 137 can include, or can optionally be combined with the subject matter of one or any combination of EMBODIMENTS 107-134, to optionally include the polymer solution further including a bioactive substance dispersed in the polymer solution.

EMBODIMENT 138 can include, or can optionally be combined with the subject matter of EMBODIMENT 137, to optionally include the biologically active substance comprising deoxyribonucleic acid (DNA).

EMBODIMENT 139 can include, or can optionally be combined with the subject matter of one or any combination of EMBODIMENTS 137 and 138, to optionally include the biologically active substance comprising ribonucleic acid (RNA).

EMBODIMENT 140 can include, or can optionally be combined with the subject matter of one or any combination of EMBODIMENTS 137-139, to optionally include the biologically active substance comprising one or more proteins.

EMBODIMENT 141 can include, or can optionally be combined with the subject matter of one or any combination of EMBODIMENTS 137-140, to optionally include the biologically active substance comprising one or more viruses.

EMBODIMENT 142 can include, or can optionally be combined with the subject matter of one or any combination of EMBODIMENTS 137-141, to optionally include the biologically active substance comprising one or more bacteria.

EMBODIMENT 143 can include, or can optionally be combined with the subject matter of one or any combination of EMBODIMENTS 137-142, to optionally include the biologically active substance comprising one or more types of mammalian cells.

EMBODIMENT 144 can include, or can optionally be combined with the subject matter of one or any combination of EMBODIMENTS 137-143, to optionally include the biologically active substance comprising one or more of DNA, RNA, one or more proteins, one or more viruses, bacteria, and mammalian cells.

EMBODIMENT 145 can include, or can optionally be combined with the subject matter of one or any combination of EMBODIMENTS 107-144, to optionally include the ceramic precursor solution comprising a silica precursor.

EMBODIMENT 146 can include, or can optionally be combined with the subject matter of one or any combination of EMBODIMENTS 107-145, to optionally include the ceramic precursor solution comprising a bioactive glass precursor.

EMBODIMENT 147 can include, or can optionally be combined with the subject matter of one or any combination of EMBODIMENTS 107-146, to optionally include the ceramic precursor solution comprising a silica precursor and a bioactive glass precursor.

EMBODIMENT 148 can include, or can optionally be combined with the subject matter of one or any combination of EMBODIMENTS 107-147, to optionally include the electrospinning device being configured to form a nanofiber or a microfiber.

EMBODIMENT 149 can include, or can optionally be combined with the subject matter of one or any combination of EMBODIMENTS 1-148, to include subject matter (such as an apparatus, a device, a method, or one or more means for performing acts), such as can include a method comprising feeding a ceramic precursor solution and a polymer solution to a microfluidic device, mixing the ceramic precursor solution and the polymer solution and reacting the ceramic precursor solution and the polymer solution in the microfluidic device to form an electrospinning solution having a predetermined viscosity, and electrospinning the electrospinning solution to form a fibrous body comprising a ceramic formed from the ceramic precursor solution and a polymer formed from the polymer solution.

EMBODIMENT 150 can include, or can optionally be combined with the subject matter of EMBODIMENT 149, to optionally include the microfluidic device comprising an adapter configured to receive the ceramic precursor from the ceramic precursor supply system and the polymer from the polymer supply system and to combine the ceramic precursor and the polymer.

EMBODIMENT 151 can include, or can optionally be combined with the subject matter of one or any combination of EMBODIMENTS 149 and 150, to optionally include mixing and reacting the ceramic precursor solution and the polymer solution comprising reacting the ceramic precursor solution and the polymer solution for a predetermined residence time.

EMBODIMENT 152 can include, or can optionally be combined with the subject matter of one or any combination of EMBODIMENTS 149-151, to optionally include the electro spinning comprising feeding the electro spinning solution to an electrospinning conduit and applying a voltage to the electrospinning conduit.

EMBODIMENT 153 can include, or can optionally be combined with the subject matter of one or any combination of EMBODIMENTS 149-152, to optionally include feeding a core solution to an inner conduit nested within the electrospinning conduit.

EMBODIMENT 154 can include, or can optionally be combined with the subject matter of one or any combination of EMBODIMENTS 149-153, to optionally include electrospinning the electrospinning solution forming a core-shell type fiber comprising a shell of the ceramic and the polymer and a core surrounded or substantially surrounded by the shell.

EMBODIMENT 155 can include, or can optionally be combined with the subject matter of EMBODIMENT 153, to optionally include the core solution comprising a core polymer solution and a biologically active substance dispersed in the polymer solution.

EMBODIMENT 156 can include, or can optionally be combined with the subject matter of EMBODIMENT 155, to optionally include the core polymer solution comprising a solution of polyvinyl alcohol.

EMBODIMENT 157 can include, or can optionally be combined with the subject matter of one or any combination of EMBODIMENTS 155 and 156, to optionally include the core polymer solution comprising a solution of polyethylene oxide.

EMBODIMENT 158 can include, or can optionally be combined with the subject matter of one or any combination of EMBODIMENTS 155-157, to optionally include the core polymer solution comprising a solution of polyethylene glycol.

EMBODIMENT 159 can include, or can optionally be combined with the subject matter of one or any combination of EMBODIMENTS 155-158, to optionally include the core polymer solution comprising a solution of gelatin.

EMBODIMENT 160 can include, or can optionally be combined with the subject matter of one or any combination of EMBODIMENTS 155-159, to optionally include the core polymer solution comprising a solution of dextran.

EMBODIMENT 161 can include, or can optionally be combined with the subject matter of one or any combination of EMBODIMENTS 155-160, to optionally include the core polymer solution comprising a solution of a carbohydrate.

EMBODIMENT 162 can include, or can optionally be combined with the subject matter of one or any combination of EMBODIMENTS 155-161, to optionally include the core polymer solution comprising a solution of one or more of polyvinyl alcohol, polyethylene oxide, polyethylene glycol, gelatin, dextran, and carbohydrate.

EMBODIMENT 163 can include, or can optionally be combined with the subject matter of EMBODIMENT 155, to optionally include the biologically active substance comprising deoxyribonucleic acid (DNA).

EMBODIMENT 164 can include, or can optionally be combined with the subject matter of one or any combination of EMBODIMENTS 155 and 164, to optionally include the biologically active substance comprising ribonucleic acid (RNA).

EMBODIMENT 165 can include, or can optionally be combined with the subject matter of one or any combination of EMBODIMENTS 155, 163, and 164, to optionally include the biologically active substance comprising one or more proteins.

EMBODIMENT 166 can include, or can optionally be combined with the subject matter of one or any combination of EMBODIMENTS 155 and 163-165, to optionally include the biologically active substance comprising one or more viruses.

EMBODIMENT 167 can include, or can optionally be combined with the subject matter of one or any combination of EMBODIMENTS 155 and 163-166, to optionally include the biologically active substance comprising one or more bacteria.

EMBODIMENT 168 can include, or can optionally be combined with the subject matter of one or any combination of EMBODIMENTS 155 and 163-167, to optionally include the biologically active substance comprising one or more types of mammalian cells.

EMBODIMENT 166 can include, or can optionally be combined with the subject matter of one or any combination of EMBODIMENTS 155 and 163-168, to optionally include the biologically active substance comprising one or more of DNA, RNA, one or more proteins, one or more viruses, bacteria, and mammalian cells.

EMBODIMENT 167 can include, or can optionally be combined with the subject matter of one or any combination of EMBODIMENTS 149-166, to optionally include the polymer solution comprising a water-soluble polymer.

EMBODIMENT 168 can include, or can optionally be combined with the subject matter of one or any combination of EMBODIMENTS 149-167, to optionally include the polymer solution comprising a solution polyvinyl alcohol.

EMBODIMENT 169 can include, or can optionally be combined with the subject matter of one or any combination of EMBODIMENTS 149-168, to optionally include the polymer solution comprising a solution of polyethylene oxide.

EMBODIMENT 170 can include, or can optionally be combined with the subject matter of one or any combination of EMBODIMENTS 149-168, to optionally include the polymer solution comprising a solution of polyethylene glycol.

EMBODIMENT 171 can include, or can optionally be combined with the subject matter of one or any combination of EMBODIMENTS 149-170, to optionally include the polymer solution comprising a solution of gelatin.

EMBODIMENT 172 can include, or can optionally be combined with the subject matter of one or any combination of EMBODIMENTS 149-171, to optionally include the polymer solution comprising a solution of dextran.

EMBODIMENT 173 can include, or can optionally be combined with the subject matter of one or any combination of EMBODIMENTS 149-172, to optionally include the polymer solution comprising a solution of a carbohydrate.

EMBODIMENT 174 can include, or can optionally be combined with the subject matter of one or any combination of EMBODIMENTS 149-173, to optionally include the polymer solution comprising a solution of one or more of polyvinyl alcohol, polyethylene oxide, polyethylene glycol, gelatin, dextran, and carbohydrate.

EMBODIMENT 175 can include, or can optionally be combined with the subject matter of one or any combination of EMBODIMENTS 149-174, to optionally include the polymer solution including a bioactive substance dispersed in the polymer solution.

EMBODIMENT 176 can include, or can optionally be combined with the subject matter of EMBODIMENT 175, to optionally include the biologically active substance comprising deoxyribonucleic acid (DNA).

EMBODIMENT 177 can include, or can optionally be combined with the subject matter of one or any combination of EMBODIMENTS 175 and 176, to optionally include the biologically active substance comprising ribonucleic acid (RNA).

EMBODIMENT 178 can include, or can optionally be combined with the subject matter of one or any combination of EMBODIMENTS 175-177, to optionally include the biologically active substance comprising one or more proteins.

EMBODIMENT 179 can include, or can optionally be combined with the subject matter of one or any combination of EMBODIMENTS 175-178, to optionally include the biologically active substance comprising one or more viruses.

EMBODIMENT 180 can include, or can optionally be combined with the subject matter of one or any combination of EMBODIMENTS 175-179, to optionally include the biologically active substance comprising one or more bacteria.

EMBODIMENT 181 can include, or can optionally be combined with the subject matter of one or any combination of EMBODIMENTS 175-180, to optionally include the biologically active substance comprising one or more types of mammalian cells.

EMBODIMENT 182 can include, or can optionally be combined with the subject matter of one or any combination of EMBODIMENTS 175-181, to optionally include the biologically active substance comprising one or more of DNA, RNA, one or more proteins, one or more viruses, bacteria, and mammalian cells.

EMBODIMENT 183 can include, or can optionally be combined with the subject matter of one or any combination of EMBODIMENTS 149-182, to optionally include the ceramic precursor solution comprising a silica precursor.

EMBODIMENT 184 can include, or can optionally be combined with the subject matter of one or any combination of EMBODIMENTS 149-183, to optionally include the ceramic precursor solution comprising a bioactive glass precursor.

EMBODIMENT 185 can include, or can optionally be combined with the subject matter of one or any combination of EMBODIMENTS 149-184, to optionally include the ceramic precursor solution comprising a silica precursor and a bioactive glass precursor.

EMBODIMENT 186 can include, or can optionally be combined with the subject matter of one or any combination of EMBODIMENTS 149-185, to optionally include the electrospinning forming a nanofiber or a microfiber.

EMBODIMENT 187 can include, or can optionally be combined with the subject matter of one or any combination of EMBODIMENTS 149-186, to optionally include the electrospinning of the electrospinning solution forming a plurality of fibrous bodies.

EMBODIMENT 188 can include, or can optionally be combined with the subject matter of EMBODIMENT 187, to optionally include forming a mat or fabric comprising the plurality of fibrous bodies.

The terms and expressions which have been employed are used as terms of description and not of limitation, and there is no intention that in the use of such terms and expressions of excluding any equivalents of the features shown and described or portions thereof, but it is recognized that various modifications are possible within the scope of the inventive subject matter claimed. Thus, it should be understood that although the embodiments of the present invention have been specifically disclosed by examples and optional features, modification and variation of the concepts herein disclosed may be resorted to by those of ordinary skill in the art, and that such modifications and variations are considered to be within the scope of the subject matter of the present invention as defined by the appended claims.

What is claimed is:

1. An ultrafine fiber comprising:
a ceramic-based fibrous body; and
a biologically active substance encapsulated in the body, substantially encapsulated in the body, or surface-attached to the body.

2. The ultrafine fiber of claim 1, wherein the body comprises a polymer and a ceramic gel network.

3. The ultrafine fiber of claim 2, wherein the polymer comprises a water-soluble polymer.

4. The ultrafine fiber of claim 2, wherein the ceramic gel network comprises at least one of a silica gel network and a bioactive glass gel network.

5. The ultrafine fiber of claim 1, wherein the fibrous body is a nanofiber or a microfiber.

6. The ultrafine fiber of claim 1, wherein the ceramic-based fibrous body comprises a core comprising the biologically active substance, and a ceramic-based shell surrounding or substantially surrounding the core.

7. The ultrafine fiber of claim 6, wherein the core comprises a core polymer encapsulating or substantially encapsulating the biologically active substance, and wherein the shell comprises a shell polymer and a ceramic gel network.

8. The ultrafine fiber of claim 7, wherein one or both of the core polymer and the shell polymer comprises a water-soluble polymer.

9. The ultrafine fiber of claim 7, wherein the ceramic gel network of the shell comprises at least one of a silica gel network and a bioactive glass gel network.

10. The ultrafine fiber of claim 1, wherein the biologically active substance comprises at least one of DNA, RNA, one or more proteins, one or more viruses, bacteria, and mammalian cells.

11. A reactive mat formed from a plurality of the ultrafine fibers of claim 1.

12. A reactive mat formed from a plurality of the ultrafine fibers of claim 6.

13. A system comprising:
a ceramic precursor supply system configured to supply a ceramic precursor solution;
a polymer supply system configured to supply a polymer solution;
a microfluidic device configured to mix and react the ceramic precursor solution and the polymer solution to form an electrospinning solution having a predetermined viscosity; and
an electrospinning device configured to electro spin the electrospinning solution to form a fibrous body comprising a ceramic formed from the ceramic precursor solution and a polymer formed from the polymer solution.

14. The system of claim 13, wherein the microfluidic device comprises an adapter configured to receive the ceramic precursor from the ceramic precursor supply system and the polymer from the polymer supply system and to combine the ceramic precursor and the polymer.

15. The system of claim 13, wherein the electrospinning device comprises:
an electrospinning conduit configured to receive the electrospinning solution;
a power supply configured to apply a voltage to the conduit; and
a conductive plate to collect the fibrous body.

16. The system of claim 15, wherein:
the electrospinning device further comprises an inner conduit nested within the electrospinning conduit;
the system further comprises a core solution supply system configured to supply a core solution to the inner conduit; and
the electrospinning device is configured to form a core-shell type fiber comprising a shell of the ceramic and the polymer and a core surrounded or substantially surrounded by the shell.

17. A method comprising:
feeding a ceramic precursor solution and a polymer solution to a microfluidic device;
mixing the ceramic precursor solution and the polymer solution and reacting the ceramic precursor solution and the polymer solution in the microfluidic device to form an electrospinning solution having a predetermined viscosity; and
electrospinning the electrospinning solution to form a fibrous body comprising a ceramic formed from the ceramic precursor solution and a polymer formed from the polymer solution.

18. The method of claim 17, wherein the microfluidic device comprises an adapter configured to receive the ceramic precursor from the ceramic precursor supply system and the polymer from the polymer supply system and to combine the ceramic precursor and the polymer.

19. The method of claim 17, wherein the electrospinning comprises feeding the electrospinning solution to an electrospinning conduit and applying a voltage to the electrospinning conduit.

20. The method of claim 19, further comprising:
feeding a core solution to an inner conduit nested within the electrospinning conduit;
wherein electrospinning the electrospinning solution forms a core-shell type fiber comprising a shell of the ceramic and the polymer and a core surrounded or substantially surrounded by the shell.

21. The method of claim 17, wherein the electrospinning the electrospinning solution forms a plurality of fibrous bodies, further comprising forming a mat or fabric comprising the plurality of fibrous bodies.
